(19) United States
(12) Patent Application Publication
(10) Pub. No.: US 2023/0248784 A1
(43) Pub. Date: Aug. 10, 2023

(54) AMMONIA OXIDIZING MICROORGANISMS FOR USE AND DELIVERY IN BUILT ENVIRONMENTS

(71) Applicant: AOBIOME LLC, Cambridge, MA (US)

(72) Inventors: Lauren Nicole Ambrogio, Boulder, CO (US); James Hoffman, Woodstock, NY (US)

(73) Assignee: AOBIOME LLC, Cambridge, MA (US)

(21) Appl. No.: 18/124,285

(22) Filed: Mar. 21, 2023

Related U.S. Application Data
(63) Continuation of application No. 17/929,184, filed on Sep. 1, 2022.
Application No. 17/929,184 is a continuation of application No. 16/723,054, filed on Dec. 20, 2019, now abandoned.
(60) Provisional application No. 62/784,596, filed on Dec. 24, 2018.

(51) Int. Cl. A61K 35/74; A01N 63/20

(52) U.S. Cl. CPC A61K 35/74; A01N 63/20

(57) ABSTRACT

Methods of treating an environmental surface by depositing a preparation including nonpathogenic bacteria on the environmental surface are disclosed. Methods of treating an environmental surface by depositing a preparation including ammonia oxidizing microorganisms on the environmental surface are disclosed. Methods of introducing nonpathogenic bacteria into a built environment are disclosed. Methods of introducing ammonia oxidizing microorganisms into a built environment are disclosed. Methods of modulating a microbiome associated with a built environment or maintaining a microbiome associated with a built environment are disclosed. Methods of treating a subject by introducing ammonia oxidizing microorganisms into the built environment are also disclosed.

### CROSS-REFERENCE TO RELATED APPLICATIONS

This application is a continuation of U.S. Application Serial No. 17/929,184 titled “AMMONIA OXIDIZING MICROORGANISMS FOR USE AND DELIVERY IN BUILT ENVIRONMENTS,” and filed Sep. 1, 2022, which is a continuation of U.S. Application Serial No. 16/723,054 titled “AMMONIA OXIDIZING MICROORGANISMS FOR USE AND DELIVERY IN BUILT ENVIRONMENTS,” and filed Dec. 20, 2019, which claims priority under 35 U.S.C. § 119(e) to U.S. Provisional Application No. 62/784,596 titled “AMMONIA OXIDIZING MICROORGANISMS FOR USE AND DELIVERY IN BUILT ENVIRONMENTS,” filed Dec. 24, 2018, the contents of which are hereby incorporated herein by reference in their entireties for all purposes.

### FIELD OF THE TECHNOLOGY

Aspects relate generally to the microbiome and, more specifically, to the restoration of nonpathogenic bacteria, e.g., beneficial bacteria to the microbiome.

### BACKGROUND

Bacteria and other microorganisms are ubiquitous in the environment. The discovery of pathogenic bacteria and the germ theory of disease have had a tremendous effect on health and disease states. Microorganisms are a normal part of the environment of all living things and may be beneficial. In the gut, for example, bacteria are not pathogenic under normal conditions, and in fact improve health by rendering the normal intestinal contents less hospitable for disease causing organisms.

### SUMMARY

In accordance with one aspect, there is provided a method of treating an environmental surface. The method may comprise depositing a preparation comprising nonpathogenic bacteria on the environmental surface, thereby treating the environmental surface.

In accordance with another aspect, there is provided a method of introducing nonpathogenic bacteria into a built environment. The method may comprise introducing a preparation comprising the nonpathogenic bacteria to a conduit in fluid communication with the built environment.

In accordance with another aspect, there is provided a method of treating an environmental surface. The method may comprise depositing a preparation comprising ammonia oxidizing microorganisms (AOM) on the environmental surface, thereby treating the environmental surface.

In accordance with another aspect, there is provided a method of modulating a microbiome associated with a built environment. The method may comprise conditioning the built environment with a preparation comprising AOM, thereby modulating the microbiome associated with the built environment.

In accordance with another aspect, there is provided a method of introducing AOM into a built environment. The method may comprise introducing a preparation comprising AOM to a conduit in fluid communication with the built environment.

In accordance with another aspect, there is provided a method of maintaining a microbiome associated with a built environment. The method may comprise introducing a preparation comprising AOM to a conduit in fluid communication with the built environment.

In accordance with another aspect, there is provided a method of treating a subject. The method may comprise introducing an effective amount of a preparation comprising AOM to a built environment of the subject, thereby treating the subject.

The environmental surface may be associated with an inanimate object. The environmental surface may relate to a surface of a building. The environmental surface may relate to a surface of a construction material. The environmental surface may relate to an article of furniture. In some embodiments, the environmental surface may relate to a surface of a fixture.

In some embodiments, a sufficient amount of the preparation comprising AOM may be deposited such that AOM are transferred to a subject, e.g. a subject whose body, e.g., skin, nose, etc., comes into contact with the environmental surface. A target percentage of administered AOM may be transferred to the subject.

In some embodiments, the preparation may modulate a microbiome or microbial community associated with or on the environmental surface.

The method may further comprise introducing a subject to the built environment.

In some embodiments, the conduit comprises an auxiliary system, e.g. an HVAC system.

In some embodiments, the conduit may comprise an appliance, e.g. a humidifier or an air conditioner, having a reservoir comprising the preparation of AOM.

The auxiliary system or appliance may be configured to provide the preparation as a spray, aerosol, or mist characterized by a predetermined particle size. The preparation may be introduced as fine particles. The particles may have a particle size range (PSR) of between about 0.1 micron and about 500 microns. At least 1, 5, 10, 20, 30, 40, 50, 60, 70, 80, 85, 90, 95, or 99% of the particles may have a PSR of between about 0.1 micron and about 10 microns.

The preparation may be introduced substantially continuously, e.g., substantially continuously during operation of the auxiliary system or appliance. In some embodiments, the preparation may be introduced periodically.

The preparation may be introduced to a defined space within the built environment. The built environment may be a substantially enclosed space.

In accordance with some embodiments, the built environment may be sterilized prior to introduction of the preparation comprising AOM. The environmental surface may be sterilized prior to depositing the preparation comprising AOM.

In some embodiments, health and/or well being of a subject may be promoted.

Introducing the preparation may comprise diffusing the preparation into the built environment.

Introducing the preparation may be associated with a desired local effect, e.g. a desired cosmetic effect or a desired therapeutic effect. Introducing the preparation may be associated with a desired systemic effect, e.g. a desired cosmetic effect or a desired therapeutic effect.

In some embodiments, administering an effective amount of the preparation may promote endothelial function. Administering the effective amount of the preparation may change or alter a level of nitrite or NO in the subject, e.g. locally or in circulation. Administering the effective amount of the preparation may modulate a microbiome associated with the subject.

The preparation may be administered prior to onset of a health condition. The preparation may be administered during incidence of a health condition. The preparation may be administered subsequent to the subsiding of a health condition. The preparation may be administered in response to a health-related symptom, trigger, or warning sign.

The method may further comprise determining whether the subject is in need of treatment for a health condition. The health condition may be an inflammatory condition.

The preparation may be administered as a solution, suspension, liquid, gel, or hydrogel.

The preparation may be formulated as an inhalable solution. The preparation may comprise a carrier, e.g., liquid or gas carrier. The preparation may be formulated to be substantially bio-compatible.

The preparation may be formulated to provide a health benefit or otherwise support a healthy lifestyle. The preparation may be formulated to provide a health benefit to at least one of the skin, scalp, gastrointestinal system, intranasal cavities, and respiratory system of the subject.

In some embodiments, the preparation may be substantially isotonic. The preparation may have a substantially physiological pH level.

The preparation may be formulated for immediate release or extended release. The preparation may be formulated to deliver nitrite or NO to a target tissue or systemically. The preparation may be formulated for transmucosal delivery and/or circulation, e.g. locally or systemically.

In accordance with certain embodiments, the method may further comprise administering a second amount of the preparation to the subject.

The preparation may be administered as part of a combination therapy. The method may further comprise administering a second treatment in combination with the preparation. The preparation may be administered for a period of time prior to initiating the second treatment. The preparation may be administered concurrently with the second treatment. The preparation may be administered for a period of time subsequent to ceasing the second treatment. The second treatment may be administered via an alternate mode of administration, e.g. orally or intranasally. In some embodiments, the subject may have a therapeutic level of a second treatment.

The preparation may be administered in conjunction with a medical approach that treats, e.g., is approved to treat or is commonly used to treat, a relevant disease or disorder, or a symptom of the relevant disease or disorder, e.g., an anti-inflammatory agent or a treatment for infection.

The preparation may be administered in conjunction with nitrite, nitrate, or NO.

In some embodiments, the effective amount may be a therapeutically effective dose of AOM. The therapeutically effective dose of AOM may be about or greater than about 1 × 10³, 10⁴, 10⁵, 10⁶, 10⁷, 10⁸, 10⁹, 10¹⁰, 10¹¹, 10¹², 10¹³, or 10¹⁴ CFU.

The preparation may be administered as an analgesic.

The preparation may be administered as a prophylactic.

In some embodiments, the preparation may be self-administered.

The preparation may be administered about 1, 2, 3, 4, 5, 6, 7, 8, 9, 10, 11, 12, 13, 14, 15, 16, 17, 18, 19, 20, 21, 22, 23, or 24 times per day. The preparation may be administered for about 1-3, 3-5, 5-7, 7-9, 5-10, 10-14, 12-18, 12-21, 21-28, 28-35, 35-42, 42-49, 49-56, 46-63, 63-70, 70-77, 77-84, or 84-91 days.

In accordance with certain embodiments, the preparation may be administered within 30, 60, 90, 120, 150, or 180 minutes of the subject waking from sleep. The preparation may be administered within 30, 60, 90, 120, 150, or 180 minutes prior to the subject sleeping. The preparation may be administered within 30, 60, 90, 120, 150, or 180 minutes of the subject eating. The preparation may be administered 30, 60, 90, 120, 150, or 180 minutes before the subject cleanses or showers.

The subject may be female.

The subject may be male.

The subject may be characterized as one of the following ethnicity/race: Asian, black or African American, Hispanic or Latino, white, or multi-racial.

The subject may be of an age of less than 1, or between 1-5, 5-10, 10-20, 20-30, 30-40, 40-50, 50-60, or over 60 years.

The subject may have a disrupted microbiome.

The subject may be healthy, e.g., physiological, normal, or non-diseased. The subject may have a healthy microbiome, e.g., physiological, normal, or non-diseased microbiome.

In some embodiments, the preparation may comprise AOM in a buffer solution, e.g., an aqueous buffer solution. The buffer solution, e.g., aqueous buffer solution, may comprise disodium phosphate and magnesium chloride, for example, 50 mM Na₂HPO₄ and 2 mM MgCl₂ in water. The buffer solution e.g., aqueous buffer solution, may consist essentially of disodium phosphate and magnesium chloride, for example, 50 mM Na₂HPO₄ and 2 mM MgCl₂ in water. The buffer solution, e.g., aqueous buffer solution, may consist of disodium phosphate and magnesium chloride, for example, 50 mM Na₂HPO₄ and 2 mM MgCl₂ in water.

The preparation may further comprise or be administered concurrently with a compound that promotes growth or metabolism of the AOM, NO production, and/or urease activity. The preparation may comprise at least one of ammonia, ammonium salts, and urea.

The preparation may comprise a controlled release material, e.g., slow release material.

The preparation may further comprise an excipient, e.g., a pharmaceutically acceptable excipient. The excipient may comprise one or more of: absorption and penetration enhancers, preservatives, antioxidants, buffers, chelating agents, ion exchange agents, solubilizing agents, suspending agents, thickeners, surfactants, wetting agents, tonicity-adjusting agents, enzyme inhibitors, and vehicle for proper drug delivery.

The preparation may further comprise one or more of antimicrobial agents, inert gases, protectants, vasoconstrictors or vasodilators, vehicle, water, a buffer system, lactated Ringer’s solution, saline, sugar, chelator, and a multi-dose preservative.

The preparation may be substantially free of other organisms.

In some embodiments, the preparation may comprise between about 1 × 10³ CFU/mL to about 1 × 10¹⁴ CFU/mL AOM. The preparation may comprise between about 1 × 10⁹ CFU/mL to about 10 × 10⁹ CFU/mL AOM.

The AOM may comprise ammonia oxidizing bacteria (AOB). The AOM may consist essentially of AOB. The AOM may consist of AOB. The AOM may comprise Nitrosomonas, Nitrosococcus, Nitrosospira, Nitrosocystis, Nitrosolobus, Nitrosovibrio, and combinations thereof. The AOM may be Nitrosomonas eutropha (N. eutropha). The AOM may be N. eutropha D23, having ATCC accession number PTA-121157. The AOM may comprise ammonia oxidizing archaea (AOA).

In accordance with certain embodiments, the AOM may be capable of converting ammonia or ammonium to nitrite at a rate of at least about 1 pmol/min/mg protein, e.g., at least about 0.1 nmol/min/mg protein.

The nonpathogenic bacteria may comprise beneficial bacteria. The nonpathogenic bacteria may comprise a selected community of species of bacteria. At least 10, 20, 30, 40, 50, 60, 70, 80, 85, 90, 95, or 99% of the nonpathogenic bacteria in the preparation consist essentially of a selected community of species of bacteria. The nonpathogenic bacteria may be selected from a nonpathogenic species in the group consisting of Prevotella, Sphingomonas, Pseudomonas, Acinetobacter, Fusobacterium, Megasphaera, Veillonella, Staphylococcus, Streptococcus, Corynebacterium, Propionibacterium, Rhodococcus, Microbacterium, Bacillus, Lactobacillus, Lactococcus, Streptomyces, Faecalibacterium, Bacteroides, or Bifidobacter, and combinations thereof.

In some embodiments, at least 1, 5, 10, 20, 30, 40, 50, 60, 70, 80, 85, 90, 95, or 99% of the nonpathogenic bacteria may be live.

The method may comprise administering AOM and other organisms, e.g., a community of organisms.

In some embodiments, the administration may occur before or after a surgical or diagnostic procedure.

In some embodiments, a biome-friendly product may be used in connection with the administered preparation comprising AOM.

In accordance with another aspect, there is provided a preparation comprising AOM for administration to a built environment.

In accordance with another aspect, there is provided a preparation comprising AOM for administration to an environmental surface.

The preparation may be packaged for single use. The preparation may be packaged for multiple use.

The preparation may comprise AOM and other organisms, e.g., a community of organisms.

In accordance with another aspect, there is provided a device configured to administer an effective amount of a preparation comprising AOM to a built environment.

In accordance with another aspect, there is provided a device configured to administer an effective amount of a preparation comprising AOM to an environmental surface.

In accordance with another aspect, there is provided a kit comprising a preparation comprising AOM.

In accordance with another aspect, there is provided an environmental surface comprising a microbial community comprising AOM deposited on the environmental surface.

In accordance with another aspect, there is provided a built environment comprising a microbial community comprising AOM introduced to the built environment.

The disclosure contemplates all combinations of any one or more of the foregoing aspects and/or embodiments, as well as combinations with any one or more of the embodiments set forth in the detailed description and any examples.

### DETAILED DESCRIPTION

In accordance with one or more embodiments, the present disclosure provides for various methods or modes of introducing nonpathogenic bacteria to a built environment. The present disclosure provides for various methods or modes of introducing nonpathogenic bacteria to a subject. These methods or modes comprise introducing nonpathogenic bacteria to a built environment, for example, a preparation, composition, formulation, or product comprising nonpathogenic bacteria. In at least some embodiments, nonpathogenic bacteria may be introduced to a subject, for example, by introduction to an environment of the subject. The nonpathogenic bacteria may therefore generally be restored to a microbiome of the subject. In at least some embodiments, nonpathogenic bacteria may comprise or consist essentially of beneficial bacteria.

Preparations, compositions, and/or formulations, e.g., including cosmetic products, therapeutic products, consumer products, non-natural products, natural products, and fortified natural products, comprising, consisting essentially of, or consisting of nonpathogenic bacteria are disclosed. These preparations, compositions, and/or formulations are disclosed herein for use in various applications, e.g., cosmetic and/or therapeutic applications. The preparations, compositions, and/or formulations may be administered to a built environment or environmental surface. The preparations, compositions, and/or formulations may be administered in an effective amount for an intended use, e.g., a cosmetic or a therapeutic application. Preparations, compositions, and/or formulations comprising nonpathogenic bacteria for various modes of administration to a subject are provided. Preparations, compositions, and/or formulations comprising nonpathogenic bacteria for use in the treatment of various conditions and/or disorders in a subject are provided. Methods of treating a subject for various conditions and/or disorders via administration of nonpathogenic bacteria are disclosed. Devices for use in administering nonpathogenic bacteria to ab built environment or environmental surface are provided. Devices for use in administering nonpathogenic bacteria to a subject are also provided. An environmental surface comprising a microbial community deposited on the environmental surface is disclosed. A built environment comprising a microbial community introduced to the built environment is also disclosed.

### Microbiology of Exemplary Nonpathogenic Bacteria

In accordance with one or more embodiments, essentially any nonpathogenic bacteria can be used or implemented. The nonpathogenic bacteria may generally be autotrophic or heterotrophic. These bacteria have beneficial properties, e.g., in connection with various cosmetic and therapeutic uses, in accordance with one or more embodiments described herein.

Nonpathogenic bacteria may be selected for meeting a predetermined criteria or reference. For example, nonpathogenic bacteria may be selected for ability to compete with pathogenic bacteria in the microbiome of a subject. In some embodiments, nonpathogenic bacteria may be selected for their ability to produce byproducts that inhibit growth or reproduction of pathogenic bacteria in the microbiome of a subject.

Certain bacteria, for example, including those commonly present in the microbiome of the lung, may inhibit the growth and reproduction of pathogenic bacteria therein. The most significant pathogenic bacteria of the lung include M. catarrhalis, H. influenzae, and S. pneumoniae. While not wishing to be bound by any particular theory, certain nonpathogenic bacteria from the respiratory microbiome may produce anti-inflammatory and antimicrobial particles, including, e.g., interleukin 10 (IL-10), FOXP3, and secretory immunoglobulin A (sIgA), and induce a Th1 response, which inhibits pathological growth.

The nonpathogenic bacteria of this disclosure may be from a genus selected from the group consisting of Prevotella, Sphingomonas, Pseudomonas, Acinetobacter, Fusobacterium, Megasphaera, Veillonella, Staphylococcus, or Streptococcus, and combinations thereof. Nonpathogenic bacteria of such genera, for example, may inhibit the growth of pathogenic bacteria by colonizing the lung or delivering one or more product or byproduct to the lung.

Likewise, nonpathogenic bacteria of the nasal microbiome may inhibit the growth and reproduction of pathogenic bacteria therein. Specifically, S. pneumoniae and S. aureus, while commonly benign when present in the nasal passage microbiome, can sometimes become pathogenic and cause severe disease. It is not well understood what causes a pathogenic state of these bacteria, however, it is generally believed that colonization is a requirement for infection. While not wishing to be bound by any particular theory, certain nonpathogenic bacteria from the nasal microbiome, for example, C. accolens, may inhibit pneumococcal growth in the microbiome by releasing fatty acids that inhibit the pathogenic bacteria. It is believed that C. accolens releases lipase LipS1 which hydrolyzes triacylglycerols, e.g., triolein, commonly found lining the nasal passages to release oleic acid, a fatty acid which inhibits pneumococcal growth.

The nonpathogenic bacteria of this disclosure may be from a genus selected from the group consisting of Staphylococcus, Corynebacterium, Propionibacterium, Rhodococcus, Microbacterium, or Streptococcus, and combinations thereof. Nonpathogenic bacteria of such genera, for example, may inhibit the growth of pathogenic bacteria by colonizing the nasal passages or delivering one or more product or byproduct to the nasal passages.

In some embodiments, the nonpathogenic bacteria may comprise one or more bacterium of the genus selected from the group consisting of Bacillus, Lactobacillus, Lactococcus, Streptomyces, Faecalibacterium, Bacteroides, or Bifidobacter and combinations thereof, e.g., L. rhamnosus, F. prausnitzii, and B. fragilis.

The nonpathogenic bacteria, e.g., ammonia oxidizing microorganism may generate nitrite and/or nitric oxide from ammonia. Properties of autotrophic ammonia oxidizing bacteria (AOB), for example, are well described by Whitlock in U.S. Pat. No. 7,820,420, incorporated by reference herein, in its entirety for all purposes. In some embodiments, reference to ammonia oxidizing bacteria may be applicable to any ammonia oxidizing microorganisms (AOM), e.g., ammonia oxidizing bacteria (AOB) and ammonia oxidizing archaea (AOA).

Without wishing to be bound to any particular theory, due to the roles of nitrite and nitric oxide as important components of several physiological functions, such as vasodilation, inflammation and wound healing, these bacteria may have various beneficial properties for both healthy and immunopathological conditions. These bacteria are safe for use in humans because they are slow-growing, cannot grow on organic carbon sources, may be sensitive to soaps and antibiotics, and have never been associated with any disease or infection in animals or humans.

Ammonia oxidizing microorganisms generate coenzyme Q 8 (CoQ8) as a byproduct of the process by which they generate nitrite and nitric oxide. CoQ8 is a coenzyme Q having 8 carbons in its isoprenoid side chain. Without wishing to be bound to any particular theory, due to the role of coenzyme Q as an important component of several cell functions, such as mediating cell signaling and preventing cell death (anti-aging), these microorganisms’ beneficial properties may further be enhanced by their specific ability to generate CoQ8.

The ammonia oxidizing bacteria of this disclosure may be from a genus selected from the group consisting of Nitrosomonas, Nitrosococcus, Nitrosospria, Nitrosocystis, Nitrosolobus, Nitrosovibrio, and combinations thereof. Examples of ammonia oxidizing bacteria include Nitrosomonas eutropha strains, e.g., D23 and C91 as discussed herein. D23 Nitrosomonas eutropha strain refers to the strain, designated AOB D23-100, deposited with the American Tissue Culture Collection (ATCC) (10801 University Blvd., Manassas, VA, USA) on Apr. 8, 2014 having accession number PTA-121157. The nucleic acid sequence(s), e.g., genome sequence, of accession number PTA-121157 are hereby incorporated herein by reference in their entireties for all purposes. “AOB D23-100” may also be referred to as D23 or B244 throughout this disclosure.

Examples of ammonia oxidizing archaea include archaea in the genera Methanobrevibacter, Methanosphaera, Methanosarcina, Nitroscaldus, Nitrosopumilus, and Nitrososphaera (e.g. Nitrososphaera viennensis, Nitrososphaera gargensis). Different phylotypes of archaea, e.g., methanogens and halphilic archaeon, may be included in the preparations disclosed herein. Examples of archaea further include archaea in the lineages of phyla Euryarchaeota (e.g. Methanosarcina), Crenarchaeota, Aigarchaeota, and Thaumarchaeota (e.g. Giganthauma karukerense, Giganthauma insulaporcus, Caldiarchaeum subterraneum, Cenarchaeum symbiosum).

This disclosure provides, inter alia, N. eutropha strain D23, a unique, e.g., optimized strain of ammonia oxidizing bacteria that can increase production of nitric oxide and nitric oxide precursors on a surface of a subject, e.g., a human subject. In certain embodiments, the N. eutropha strain comprises a nucleic acid sequence, e.g., a genome, that hybridizes to SEQ ID NO: 1 of International (PCT) Patent Application Publication No. WO2015160911 (International (PCT) Patent Application Serial No. PCT/US2015/025909 filed on Apr. 15, 2015, incorporated by reference herein, in its entirety for all purposes), or to the genome of the D23 strain deposited in the form of 25 vials with the ATCC patent depository on Apr. 8, 2014, designated AOB D23-100, under accession number PTA-121157, or their complements, under low stringency, medium stringency, high stringency, or very high stringency, or other hybridization condition.

The practice of the present invention may employ, unless otherwise indicated, conventional methods of immunology, molecular biology, and recombinant DNA techniques within the skill of the art. Such techniques are explained fully in the literature. See, e.g., Sambrook, et al. Molecular Cloning: A Laboratory Manual (Current Edition); and Current Protocols in Molecular Biology (F.M. Ausubel, et al. eds., current edition).

### Microbiology of Ammonia Oxidizing Microorganisms (AOM)

In accordance with one or more embodiments, essentially any ammonia oxidizing microorganism (AOM) can be used or implemented. The ammonia oxidizing microorganisms may generally be autotrophic. The ammonia oxidizing microorganisms may generate nitrite and/or nitric oxide from ammonia.

Properties of autotrophic ammonia oxidizing bacteria (AOB), for example, are well described by Whitlock in U.S. Pat. No. 7,820,420. Since that filing, the class of autotrophic microorganisms that oxidize ammonia for ATP production has been expanded to encompass ammonia oxidizing archaea (AOA), and archaea have been moved out of the class of bacteria and into their own distinct class. For the purposes of this disclosure, any and all autotrophic ammonia oxidizing microorganisms that share the properties of oxidation of ammonia to generate ATP can be implemented. AOM, including both AOB and AOA, share the necessary properties of oxidation of ammonia into NO and nitrite and all known AOM lack capacity for virulence because of their inability to use organic substrates for ATP generation. Bacteria can utilize ammonia at higher concentrations, while archaea can utilize ammonia at lower concentrations. Physiological levels of ammonia are within the range that both bacteria (AOB) and archaea (AOA) can utilize. Any reference specifically to ammonia oxidizing bacteria throughout this disclosure should be considered equally applicable to any ammonia oxidizing microorganism, e.g., any ammonia oxidizing archaea, and these terms may all be used interchangeably herein.

Ammonia oxidizing bacteria (AOB) are ubiquitous Gram-negative obligate bacteria with a unique capacity to generate energy exclusively from the conversion of ammonia to nitrite. In some embodiments, ammonia oxidizing bacteria (AOB) of the genus Nitrosomonas are Gram-negative obligate autotrophic (chemolithoautotrophic) bacteria with a unique capacity to generate nitrite and nitric oxide exclusively from ammonia as an energy source. They are widely present both in soil and water environments and are essential components of environmental nitrification processes. These bacteria have beneficial properties, e.g., in connection with various cosmetic and therapeutic uses, in accordance with one or more embodiments described herein. Without wishing to be bound to any particular theory, due to the roles of nitrite and nitric oxide as important components of several physiological functions, such as vasodilation, inflammation and wound healing, these bacteria may have various beneficial properties for both healthy and immunopathological conditions. These bacteria are safe for use in humans because they are slow-growing, cannot grow on organic carbon sources, may be sensitive to soaps and antibiotics, and have never been associated with any disease or infection in animals or humans.

Ammonia oxidizing microorganisms generate coenzyme Q 8 (CoQ8) as a byproduct of the process by which they generate nitrite and nitric oxide. CoQ8 is a coenzyme Q having 8 carbons in its isoprenoid side chain. Without wishing to be bound to any particular theory, due to the role of coenzyme Q as an important component of several cell functions, such as mediating cell signaling and preventing cell death (anti-aging), these microorganisms’ beneficial properties may further be enhanced by their specific ability to generate CoQ8.

In some embodiments, ammonia oxidizing bacteria may catalyze the following reactions.

At a neutral pH level, ammonia generated from ammonium around neutral pH conditions is the substrate of the initial reaction. The conversion of ammonia to nitrite takes place in two steps catalyzed respectively by ammonia monooxygenase (AMO) and hydroxylamine oxidoreductase (HAO), as follows:

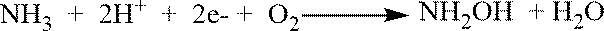

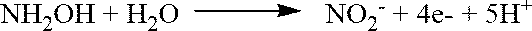

In some instances, reaction B is reported as follows, to indicate nitrous acid (HNO₂) formation at low pH:

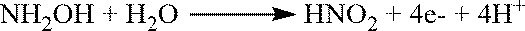

In certain embodiments, NH₄⁺ and NH₃ may be used interchangeably throughout the disclosure.

Examples of ammonia oxidizing bacteria include Nitrosomonas eutropha strains, e.g., D23 and C91 as discussed herein, and other bacteria in the genera Nitrosomonas, Nitrosococcus, Nitrosospira, Nitrosocystis, Nitrosolobus, and Nitrosovibrio. D23 Nitrosomonas eutropha strain refers to the strain, designated AOB D23-100, deposited with the American Tissue Culture Collection (ATCC) (10801 University Blvd., Manassas, VA, USA) on Apr. 8, 2014 having accession number PTA-121157. The nucleic acid sequence(s), e.g., genome sequence, of accession number PTA-121157 are hereby incorporated herein by reference in their entireties for all purposes. “AOB D23-100” may also be referred to as D23 or B244 throughout this disclosure.

Examples of ammonia oxidizing archaea include archaea in the genera Methanobrevibacter, Methanosphaera, Methanosarcina, Nitroscaldus, Nitrosopumilus, and Nitrososphaera (e.g. Nitrososphaera viennensis, Nitrososphaera gargensis). Different phylotypes of archaea, e.g., methanogens and halphilic archaeon, may be included in the preparations disclosed herein. Examples of archaea further include archaea in the lineages of phyla Euryarchaeota (e.g. Methanosarcina), Crenarchaeota, Aigarchaeota, and Thaumarchaeota (e.g. Giganthauma karukerense, Giganthauma insulaporcus, Caldiarchaeum subterraneum, Cenarchaeum symbiosum).

Each and every nucleic acid sequence and amino acid sequence disclosed in International (PCT) Patent Application Publication No. WO2015/160911 (International (PCT) Patent Application Serial No. PCT/US2015/025909 as filed on Apr. 15, 2015), is hereby incorporated herein by reference in its entirety for all purposes. Likewise, any ammonia oxidizing bacteria disclosed in International (PCT) Patent Application Publication No. WO2015/160911 (International (PCT) Patent Application Serial No. PCT/US2015/025909 as filed on Apr. 15, 2015), is also hereby incorporated herein by reference in its entirety for all purposes. In certain embodiments, the ammonia oxidizing microorganism is a strain as described therein.

In accordance with one or more embodiments, ammonia oxidizing microorganisms may exist in several metabolic states, e.g. growth state, storage state, and/or polyphosphate loading state.

In accordance with one or more embodiments, ammonia oxidizing microorganisms may have desirable properties, e.g., optimized properties, such as the ability to suppress growth of pathogenic bacteria, and an enhanced ability to produce nitric oxide and nitric oxide precursors.

Optimized Nitrosomonas eutropha (N. eutropha), as that term is used herein, refers to an N. eutropha having an optimized growth rate; an optimized NH₄⁺ oxidation rate; and/or optimized resistance to NH₄⁺. In an embodiment it differs from naturally occurring N. eutropha by at least one nucleotide, e.g., a nucleotide in a gene selected from ammonia monooxygenase, hydroxylamine oxidoreductase, cytochrome c554, and cytochrome c_{M}552. The difference can arise, e.g., through selection of spontaneously arising mutation, induced mutation, or directed genetic engineering, of the N. eutropha. In an embodiment it differs from a naturally occurring N. eutropha in that it has a constellation of alleles, not present together in nature. These differences may provide for one or more of a treatment or prevention of a disease or condition, such as but not limited to one associated with low nitrite levels.

Any ammonia oxidizing bacteria, e.g., N. eutropha, for example N. eutropha referred to as “D23”, also known as “B244” or “AOB D23-100” may have several of the above-described properties. Any ammonia oxidizing archaea (AOA) may also have several of the above-described properties.

The AOBs contemplated in this disclosure may comprise mutations relative to wild-type AOBs. These mutations may, e.g., occur spontaneously, be introduced by random mutagenesis, or be introduced by targeted mutagenesis. For instance, the AOBs may lack one or more genes or regulatory DNA sequences that wild-type AOBs typically comprise. The AOBs may also comprise point mutations, substitutions, insertions, deletions, and/or rearrangements relative to the sequenced strain or a wild-type strain. The AOBs may be a purified preparation of optimized AOBs.

In certain embodiments, the AOBs are transgenic. For instance, it may comprise one or more genes or regulatory DNA sequences that wild-type ammonia oxidizing bacteria lacks. More particularly, the ammonia oxidizing bacteria may comprise, for instance, a reporter gene, a selective marker, a gene encoding an enzyme, or a promoter (including an inducible or repressible promoter). In some embodiments the additional gene or regulatory DNA sequence is integrated into the bacterial chromosome; in some embodiments the additional gene or regulatory DNA sequence is situated on a plasmid.

In some embodiments, the AOBs differ by at least one nucleotide from naturally occurring bacteria. For instance, the AOBs may differ from naturally occurring bacteria in a gene or protein that is part of a relevant pathway, e.g., an ammonia metabolism pathway, a urea metabolism pathway, or a pathway for producing nitric oxide or nitric oxide precursors. More particularly, the AOBs may comprise a mutation that elevates activity of the pathway, e.g., by increasing levels or activity of an element of that pathway.

The above-mentioned mutations can be introduced using any suitable technique. Numerous methods are known for introducing mutations into a given position. For instance, one could use site-directed mutagenesis, oligonucleotide-directed mutagenesis, or site-specific mutagenesis. Non-limiting examples of specific mutagenesis protocols are described in, e.g., Mutagenesis, pp. 13.1-13.105 (Sambrook and Russell, eds., Molecular Cloning A Laboratory Manual, Vol. 3, 3.sup.rd ed. 2001). In addition, non-limiting examples of well-characterized mutagenesis protocols available from commercial vendors include, without limitation, Altered Sites.RTM. II in vitro Mutagenesis Systems (Promega Corp., Madison, Wis.); Erase-aBase.RTM. System (Promega, Madison, Wis.); GeneTailor.TM. Site-Directed Mutagenesis System (Invitrogen, Inc., Carlsbad, Calif.); QuikChange.RTM. II Site-Directed Mutagenesis Kits (Stratagene, La Jolla, Calif.); and Transformer.TM. Site-Directed Mutagenesis Kit (BD-Clontech, Mountain View, Calif.).

In certain embodiments of the disclosure, the ammonia oxidizing microorganisms may be axenic. The preparation (formulation or composition) of ammonia oxidizing microorganisms may comprise, consist essentially of, or consist of axenic ammonia oxidizing microorganisms.

The ammonia oxidizing bacteria of this disclosure may be from a genus selected from the group consisting of Nitrosomonas, Nitrosococcus, Nitrosospria, Nitrosocystis, Nitrosolobus, Nitrosovibrio, and combinations thereof.

This disclosure provides, inter alia, N. eutropha strain D23, a unique, e.g., optimized strain of ammonia oxidizing bacteria that can increase production of nitric oxide and nitric oxide precursors on a surface of a subject, e.g., a human subject. This disclosure also provides methods of administering and using the bacteria and preparations, compositions, formulations, and products, comprising the bacteria.

In embodiments, the ammonia oxidizing bacteria, e.g., N. eutropha is non-naturally occurring. For instance, it may have accumulated desirable mutations during a period of selection. In other embodiments, desirable mutations may be introduced by an experimenter. In some embodiments, the N. eutropha may be a purified preparation, and may be an optimized N. eutropha.

In preferred embodiments, the N. eutropha strain is autotrophic and so incapable of causing infection. A preferred strain utilizes urea as well as ammonia, so that hydrolysis of the urea in sweat would not be necessary prior to absorption and utilization by the bacteria. Also, in order to grow at low pH, the bacteria may either absorb NH₄⁺ ions or urea. The selected strain should also be capable of living on the external skin of a subject, e.g., a human, and be tolerant of conditions there.

Although this disclosure refers to N. eutropha strain D23 in detail, the preparations, methods, compositions, treatments, formulas and products may be used with one or more of: one or more other strains of N. eutropha, one or more other species of Nitrosomonas, and one or more other ammonia oxidizing microorganism, e.g. ammonia oxidizing bacteria or other ammonia oxidizing archaea.

In certain embodiments, a bacterium with the above-mentioned sequence characteristics has one or more of (1) an optimized growth rate as measured by doubling time, (2) an optimized growth rate as measured by OD600, (3) an optimized NH₄⁺ oxidation rate, (4) an optimized resistance to NH₄⁺, and (4) an optimized resistance to NO₂⁻. Particular nonlimiting subcombinations of these properties are specified in the following paragraph.

In some embodiments, the ammonia oxidizing bacteria, e.g., the N. eutropha described herein, or an axenic composition thereof, has one or more of: (1) an optimized growth rate as measured by doubling time, (2) an optimized growth rate as measured by OD600, (3) an optimized NH₄⁺ oxidation rate, (4) an optimized resistance to, NH₄⁺, and (4) an optimized resistance to, NO₂⁻. For instance, the bacterium may have properties (1) and (2); (2) and (3); (3) and (4); or (4) and (5) from the list at the beginning of this paragraph. As another example, the bacterium may have properties (1), (2), and (3); (1), (2), and (4); (1), (2), and (5); (1), (3), and (4); (1), (3), and (5); (1), (4), and (5); (2), (3), and (4); (2), (3), and (5), or (3), (4), and (5) from the list at the beginning of this paragraph. As a further example, the bacterium may have properties (1), (2), (3), and (4); (1), (2), (3), and (5); (1), (2), (4), and (5); (1), (3), (4), and (5); or (2), (3), (4), and (5) from the list at the beginning of this paragraph. In some embodiments, the bacterium has properties (1), (2), (3), (4), and (5) from the list at the beginning of this paragraph.

In certain embodiments, the N. eutropha strain comprises a nucleic acid sequence, e.g., a genome, that hybridizes to SEQ ID NO: 1 of International (PCT) Patent Application Publication No. WO2015160911 (International (PCT) Patent Application Serial No. PCT/US2015/025909 filed on Apr. 15, 2015), or to the genome of the D23 strain deposited in the form of 25 vials with the ATCC patent depository on Apr. 8, 2014, designated AOB D23-100, under accession number PTA-121157, or their complements, under low stringency, medium stringency, high stringency, or very high stringency, or other hybridization condition.

The D23 strain is not believed to be a product of nature, but rather has acquired certain mutations and characteristics during an extended period of culture and selection in the laboratory. For instance, D23 has an ability to grow in conditions of greater than about 200 or 250 mM NH₄⁺ for more than 24 hours.

In some embodiments, the N. eutropha disclosed herein differ from naturally occurring bacteria in the abundance of siderophores. For instance, the N. eutropha may have elevated or reduced levels of siderophores compared to N. eutropha C91. Generally, siderophores are secreted iron-chelating compounds that help bacteria scavenge iron from their environment. Some siderophores are peptides, and others are small organic molecules.

The practice of the present invention may employ, unless otherwise indicated, conventional methods of immunology, molecular biology, and recombinant DNA techniques within the skill of the art. Such techniques are explained fully in the literature. See, e.g., Sambrook, et al. Molecular Cloning: A Laboratory Manual (Current Edition); and Current Protocols in Molecular Biology (F.M. Ausubel, et al. eds., current edition).

### Select Definitions

Throughout this disclosure, formulation may refer to a composition or preparation or product.

A nonpathogenic bacterium refers to a bacterium of a species which generally does not cause harm, disease, or death to the subject. Nonpathogenic bacteria may include non-harmful and non-virulent bacteria. The nonpathogenic property of the bacterium may be specific to a given subject. For instance, a particular strain of bacteria may be pathogenic to certain a subject, e.g., a human, but not other classes of subjects, e.g., non-human mammals. Some typically nonpathogenic bacteria may become pathogenic after entering the body, e.g., through ingestion, or after multiplying to a certain number of viable bacteria (CFU). In some embodiments, “nonpathogenic bacteria” refers to bacterial strains which are typically nonpathogenic. In some embodiments, “nonpathogenic bacteria” refers to bacterial strains which are not known to cause harm. In some embodiments, nonpathogenic bacteria are not known to cause harm, disease, or death to the subject.

Nonpathogenic bacteria may include beneficial bacteria. A beneficial bacterium refers to a live bacterium which may confer a health benefit on the subject. Beneficial bacteria may be associated with a subject’s microbiome, e.g., providing a benefit to a subject’s microbiome. For example, beneficial bacteria may compete with pathogenic bacteria, e.g., consuming scarce nutrients, or generating byproducts that are harmful to other organisms, e.g., changing a pH level that is not conducive to the undesirable organism’s growth. Beneficial bacteria may provide a benefit by delivering a beneficial product or byproduct to the subject, e.g., a product or byproduct which typically inhibits growth or reproduction of pathogenic bacteria. Beneficial bacteria may additionally or alternatively deliver a product or byproduct which promotes growth and metabolism of other beneficial bacteria.

As used herein, “axenic” refers to a composition comprising an organism that is substantially free of other organisms. For example, an axenic culture of nonpathogenic bacteria is a culture that is substantially free of organisms other than nonpathogenic bacteria. For example, an axenic culture of a selected community of species of bacteria is a culture that is substantially free of organisms other than bacterial species from the selected community. In some embodiments, “substantially free” denotes undetectable by a method used to detect other organisms, e.g., plating the culture and examining colony morphology, or PCR for a conserved gene such as 16S RNA. An axenic composition may comprise elements that are not organisms, e.g., it may comprise nutrients or excipients. Any embodiment, preparation, composition, or formulation of ammonia oxidizing bacteria discussed herein may comprise, consist essentially of, or consist of optionally axenic nonpathogenic bacteria.

As used herein, “purified” refers to a composition comprising a predetermined concentration of a selected organism or community of organisms as measured against other organisms in the composition. For example, a purified culture of nonpathogenic bacteria may contain a predetermined concentration of nonpathogenic bacteria. The remainder may be organisms other than nonpathogenic bacteria, e.g., pathogenic bacteria or other organisms. In some embodiments, a purified composition may comprise elements that are not organisms in varying concentrations without affecting the “purity” of a selected organism or community of organisms. Compositions disclosed herein may be purified to at least 10, 20, 30, 40, 50, 60, 70, 80, 90, 95, or 99%. Compositions may be purified to at least 99, 99.9, 99.99, or 99.999%. Compositions may be purified to be substantially free of other organisms or to wherein substantially all of the organisms in the composition are a selected organism or community of organisms. For example, compositions can be purified to a predetermined concentration of nonpathogenic bacteria, live bacteria, isolated species of bacteria, a selected community of species of bacteria, and combinations thereof. Compositions may be purified to exclude a selected organism or community of organisms. For example, compositions disclosed herein may be substantially free of pathogenic bacteria, non-live bacteria, ammonia oxidizing bacteria, and combinations thereof.

As used herein, an “autotroph”, e.g., an autotrophic bacterium, is any organism capable of self-nourishment by using inorganic materials as a source of nutrients and using photosynthesis or chemosynthesis as a source of energy. Autotrophic bacteria may synthesize organic compounds from carbon dioxide and ATP derived from other sources, oxidation of ammonia to nitrite, oxidation of hydrogen sulfide, and oxidation of Fe²⁺ to Fe³⁺. Autotrophic bacteria of the present disclosure are incapable of causing infection.

As used herein, a “heterotroph”, e.g., a heterotrophic bacterium, is any organism which consumes organic matter as a source of nutrients and energy. Heterotrophic bacteria may consume organic carbon (e.g., glucose) or organic chemicals (e.g., carbohydrates, lipids, or proteins) for nourishment. Heterotrophic bacteria of the present disclosure may be incapable of causing infection.

Administered “in combination,” as used herein, means that two (or more) different treatments are delivered to the subject during the course of the subject’s affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concomitant” or “concurrent delivery”. In other embodiments, the delivery of one treatment ends before the delivery of the other treatment begins. This is sometimes referred to herein as “successive” or “sequential delivery.” In embodiments of either case, the treatment is more effective because of combined administration. For example, the second treatment is a more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the first treatment delivered is still detectable when the second is delivered. In some embodiments, one or more treatment may be delivered prior to diagnosis of the patient with the disorder.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “optimized growth rate” refers to one or more of: a doubling time of less than about 4, 5, 6, 7, 8, 9, or 10 hours when cultured under batch conditions as described herein; a doubling time of less than about 16, 18, 20, 22, 24, or 26 hours, when grown under chemostat conditions as described herein; or growing from an OD600 of about 0.15 to at least about 0.3, 0.4, 0.5, 0.6, 0.7, or 0.8 over about 1 or 2 days. In an embodiment, optimized growth rate is one having a doubling time that it is at least 10, 20, 30, 40, or 50% shorter than that of a naturally occurring bacterium.

As used herein, “transgenic” means comprising one or more exogenous portions of DNA. The exogenous DNA is derived from another organism, e.g., another bacterium, a bacteriophage, an animal, or a plant.

As used herein, treatment of a disease or condition refers to reducing the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein a therapeutically effective amount refers to a dose sufficient to prevent advancement, or to cause regression of a disease or condition, or which is capable of relieving a symptom of a disease or condition, or which is capable of achieving a desired result. A therapeutically effective dose can be measured, for example, as a number of bacteria or number of viable bacteria (e.g., in CFUs) or a mass of bacteria (e.g., in milligrams, grams, or kilograms), or a volume of bacteria (e.g., in mm³).

As used herein, the term “viability” refers to a nonpathogenic bacteria’s ability to provide a beneficial effect, for example, a beneficial bacteria’s ability to inhibit growth or reproduction of pathogenic bacteria, a beneficial bacteria’s ability to produce byproducts that inhibit growth or reproduction of pathogenic bacteria, or an autotrophic bacteria’s, e.g., ammonia oxidizing bacteria’s, ability to oxidize ammonia, ammonium, or urea to nitrite at a pre-determined rate.

As used herein, a “subject” may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

“Microbiome” refers to a population, e.g., one or more microorganisms that live on a surface of a subject, e.g., in the gut, mouth, skin, and/or elsewhere in a subject. The population may have one or more beneficial functions and/or benefits, relevant to supporting the life of a subject.

“Biome-friendly” refers to something, e.g., a product, e.g., a cosmetic product, e.g., a finished cosmetic product that may allow for minimal disruption of a microbiome of a subject. For example, biome-friendly refers to a product that may be applied to a subject that may allow the microbiome at the point of application to be maintained, minimally disrupted, and/or able to return to the microbiome after a period of time after application of the product. In embodiments, biome-friendly may refer to ammonia oxidizing microorganism-friendly, e.g. ammonia oxidizing bacteria- friendly in that the product may allow for minimal disruption of the ammonia oxidizing bacteria of a subject. In embodiments, “biome-friendly” may be referred to as “biome-compatible.”

As used herein, “presence” or “level” may refer to a qualitative or quantitative amount of a component, e.g., any one or more of a nonpathogenic bacterium, a beneficial bacterium, an ammonia oxidizing microorganism, ammonia, ammonium ions, urea, nitrite, nitric oxide, Th1, IL-10, FOXP3, sIgA, antipneumococcal free fatty acids, oleic acid, triolein, or lipase LipS1. The presence or level may include a zero value or a lack of presence of a component.

As used herein, the term “surfactant”, includes compounds that may lower the surface tension, or interfacial tension, between two liquids or between a liquid and a solid. Surfactants may act as detergents, wetting agents, emulsifiers, foaming agents, and dispersants. Surfactants may include one or more of the following, alone, or in combination with those listed, or other surfactants or surfactant-like compounds: cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner’s Castile soap, Dr. Bronner’s baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K), and combinations thereof. Dr. Bronner’s Castile soap and baby soap comprises water, organic coconut oil, potassium hydroxide, organic olive oil, organic fair deal hemp oil, organic jojoba oil, citric acid, and tocopherol. Surfactants may include Sodium Laurylglucosides Hydroxypropylsulfonate (Suga®nate 160NC), lauramidopropyl betaine (Cola®Teric LMB); Cocamidopropyl hydroxysultaine (Cola®Teric CBS); disodium cocoamphodiacetate (Cola®Teric CDCX-LV); sodium laurylglucosides hydroxypropyl phosphate (Suga®Fax D12). Surfactants may include sodium lauroyl methyl isethionate (Iselux® LQ-CLR-SB); sodium methyl cocoyl taurate (Pureact WS Conc.); Aqua (and) Sodium Lauroyl Methyl Isethionate (and) Cocamidopropyl Betaine (and) Sodium Cocoyl Isethionate (and) Sodium Methyl Oleoyl Taurate (Iselux ®SFS-SB). Other surfactants are contemplated by this disclosure.

A “natural product” is or may comprise a product that may be at least partially derived from nature. It may be anything or comprise anything produced by a living organism, and may include organisms themselves. Natural products may include or comprise an entire organism, and part of an organism (e.g., a leaf of a plant), an extract from an organism, an organic compound from an organism, a purified organic compound from an organism. Natural products may be or comprise organic substances found and cells, including primary metabolites (amino acids, carbohydrates, and nucleic acids) and secondary metabolites (organic compounds found in a limited range of species, e.g., polyketides, fatty acids, terpenoids, steroids, phenylpropanoids, alkaloids, specialized amino acids and peptides, specialized carbohydrates). Natural products may be or comprise polymeric organic materials such as cellulose, lignin, and proteins.

An ammonia oxidizing microorganism, e.g., ammonia oxidizing bacteria, refers to a microorganism capable of oxidizing ammonia or ammonium to nitrite at a rate, e.g., a substantial rate, e.g., a pre-determined rate. The rate, e.g., a pre-determined rate, may refer to the conversion of ammonium ions (NH₄⁺) (e.g., at about 200 mM) to nitrite (NO₂⁻), for example, as determined or measured in an in vitro assay or when administered to a subject, e.g., a human. The rate may be a conversion at a rate of at least about 1 picomole per minute per mg protein, 0.01, 0.1, 1, 10, 25, 50, 75, 125, or 150 nanomoles NO₂⁻ per minute per mg protein, e.g., about 0.01-1, 0.1-50, 50-100, 100-150, 75-175, 75-125, 100-125, 125-150, or 125-175 nanomoles/minute/mg protein, e.g., about 125 nanomoles NO₂⁻ per minute per mg protein for a continuous culture, for example having an OD of about 0.5. The rate of conversion may be between about 1 picomole per minute per mg protein to about 1 millimole per minute per mg protein. The rate of conversion may be at most about 1 mole NO₂⁻ per minute per mg protein, e.g. at least about, about, or at most about 1 decimole, 1 centimole, 1 millimole, or 1 micromole NO₂⁻ per minute per mg protein.

As used herein, “optimized NH₄⁺ oxidation rate” refers to a rate of at least about 50, 75, 125, or 150 micromoles per minute of converting NH₃ or NH₄⁺ into NO₂⁻. For instance, the rate may be at least about 50, 75, 125, or 150 micromoles per minute of converting NH₄⁺ (e.g., at about 200 mM) to NO₂⁻. In some embodiments, the rate of converting NH₃ or NH₄⁺ into NO₂⁻ may be at least about 1 pmol/min/mg protein, e.g., at least about 0.1 nmol/min/mg protein. In an embodiment, an optimized NH₄⁺ oxidation rate is one in which NH₃ or NH₄⁺ is converted into NO₂^{-,} at least 10, 20, 30, 40, or 50% more rapidly than is seen with a naturally occurring bacteria, e.g., N. eutropha.

In some embodiments, the rate refers to the conversion of ammonium ions (NH₄⁺) (e.g., at about 200 mM) to nitrite (NO₂⁻) at a rate of at least about 1 picomole, 0.01, 0.1, 1, 10, 25, 50, 75, 125, or 150 nanomoles NO₂⁻ per minute, e.g., about 0.01-1, 0.1-50, 50-100, 100-150, 75-175, 75-125, 100-125, 125-150, or 125-175 nanomoles/minute, e.g., about 125 nanomoles NO₂⁻ per minute. The rate of conversion may be at most about 1 mole NO₂⁻ per minute, e.g. at least about, about, or at most about 1 decimole, 1 centimole, 1 millimole, or 1 micromole NO₂⁻ per minute. Viable ammonia oxidizing microorganisms may generally comprise culturable AOMs or AOMs that are otherwise able to generate NO, nitrate, or nitrite.

As used herein, “optimized resistance to NH₄⁺” refers to an ability to grow in conditions of greater than 50, 75, 100, 125, 150, 175, 200, 225, 250, 275, or 300 mM NH₃ or NH₄⁺ for at least about 24 or 48 hours. In an embodiment, an optimized resistance to NH₄⁺ refers to the ability to grow at least 10, 20, 30, 40, or 50% more rapidly, or at least 10, 20, 30, 40, or 50% longer, in the presence of a selected concentration of NH₃ or NH₄⁺ than can a naturally occurring N. eutropha.

### Preparations, Compositions, Formulations, and Products Comprising Nonpathogenic Bacteria

The present disclosure provides, inter alia, compositions comprising nonpathogenic bacteria, preparations, e.g., purified and/or optimized preparations, comprising nonpathogenic bacteria, formulations comprising nonpathogenic bacteria, and various products comprising nonpathogenic bacteria, e.g., a natural product, a non-natural product, a fortified natural product, a consumer product, a therapeutic product, or a cosmetic product. The terms preparation, composition, formulation, and product may be used interchangeably herein.

Any embodiment, preparation, composition, formulation, or product of nonpathogenic bacteria discussed herein may comprise, consist essentially of, or consist of (optionally axenic) nonpathogenic bacteria, e.g., live nonpathogenic bacteria or live beneficial bacteria. In some embodiments, preparations, compositions, formulations, or product of nonpathogenic bacteria discussed herein are substantially free of other organisms. Preparations, compositions, formulations, or product of nonpathogenic bacteria discussed herein may be substantially free of non-living matter.

The preparation may comprise or be supplemented with a product or byproduct of nonpathogenic bacteria. The preparation may comprise a product or byproduct including, e.g., Th1, IL-10, FOXP3, sIgA. The preparation may comprise a product or byproduct including, e.g., antipneumococcal free fatty acids, oleic acid, triolein, or lipase LipS1. In some embodiments, the preparation may comprise or be supplemented with a product or byproduct of an ammonia oxidizing microorganism, e.g., nitrite, nitrate, nitric oxide, CoQ8. In at least some embodiments, the preparation may comprise or be supplemented with a composition that promotes growth or metabolism of nonpathogenic bacteria, promotes production of products or byproducts of nonpathogenic bacteria, promotes activity of nonpathogenic bacteria, or has a synergistic effect with nonpathogenic bacteria. For example, the preparation may comprise or be supplemented with a composition that promotes urease activity, or has a synergistic effect with ammonia oxidizing microorganisms, e.g., ammonia, ammonium salts, urea, and urease. For instance, the preparation may be supplemented with one or more of NO, nitrite, nitrate, CoQ8, ammonia, ammonium salts, urea, and urease. The supplement may be comprised in the same formulation as the nonpathogenic bacteria or in a separate formulation for concurrent or combination administration. The supplement formulation may be prepared for delivery via any delivery mode, for example via inhalation or nebulization. The preparation may comprise or be supplemented with an anti-inflammatory agent or a composition that provides an anti-inflammatory effect. For example, the preparation may comprise or be supplemented with a steroid.

The present disclosure provides for preparations comprising nonpathogenic bacteria for cosmetic use.

The present disclosure provides for preparations comprising nonpathogenic bacteria for therapeutic use.

In some embodiments, a preparation of nonpathogenic bacteria may comprise a concentration or amount, e.g., an effective amount, of nonpathogenic bacteria sufficient to have a desired cosmetic effect. The preparation may be formulated and/or delivered to impart the desired cosmetic effect locally and/or systemically.

In some embodiments, a preparation of nonpathogenic bacteria may comprise a concentration or amount, e.g., an effective amount, of nonpathogenic bacteria sufficient to have a desired therapeutic effect, e.g., to at least partially treat a condition or disease. The preparation may be formulated and/or delivered to impart the desired therapeutic effect locally and/or systemically.

In some embodiments, a preparation of nonpathogenic bacteria may comprise a concentration or amount, e.g., an effective amount, of nonpathogenic bacteria sufficient to alter, e.g., reduce or increase, an amount, concentration or proportion of a bacterium, or genus of bacteria in a subject. The bacteria may be beneficial bacteria.

In some embodiments, a preparation of nonpathogenic bacteria may comprise a concentration or amount, e.g., an effective amount, of nonpathogenic bacteria sufficient to modulate a microbiome associated with a subject.

In some embodiments, a preparation of nonpathogenic bacteria may comprise a concentration or amount, e.g., an effective amount, of nonpathogenic bacteria sufficient to modulate a microbiome, e.g., gastrointestinal, respiratory, or skin microbiome, of the subject. In some embodiments, a preparation of nonpathogenic bacteria may comprise a concentration or amount, e.g., an effective amount, of nonpathogenic bacteria to promote nonpathogenic bacterial colonization of a microbiome of the subject. For instance, a preparation of nonpathogenic bacteria may comprise a concentration or amount, e.g., an effective amount, of nonpathogenic bacteria such that when administered, the preparation inhibits growth or reproduction of pathogenic bacteria. A preparation of nonpathogenic bacteria may comprise a concentration or amount, e.g., an effective amount, of nonpathogenic bacteria to reduce a state of inflammation or promote endothelial function.

In some embodiments, a preparation of nonpathogenic bacteria may comprise a concentration or amount, e.g., an effective amount, of nonpathogenic bacteria such that when administered the nonpathogenic bacteria compete with pathogenic bacteria in the microbiome of the subject. The present disclosure provides, inter alia, non-limiting compositions comprising nonpathogenic bacteria, e.g., beneficial bacteria, e.g., a purified preparation of beneficial bacteria. In some embodiments, the beneficial bacteria in the compositions are capable of inhibiting pathogenic bacterial growth, e.g., associated with M. catarrhalis, H. influenzae, S. pneumonia, or S. aureus.

In some embodiments, a preparation of nonpathogenic bacteria may comprise a concentration or amount, e.g., an effective amount, of nonpathogenic bacteria sufficient to deliver a product of the nonpathogenic bacteria to a microbiome of the subject. The present disclosure provides, inter alia, non-limiting compositions comprising nonpathogenic bacteria, e.g., C. accolens, e.g., a purified preparation of C. accolens. In some embodiments, the C. accolens in the compositions has at least one property selected from an optimized growth rate and an optimized triacylglycerol hydrolization rate.

This disclosure also provides a composition having, e.g., L. rhamnosus, and one other type of organism, and no other types of organism. In other examples, the composition has, e.g., L. rhamnosus, and 2, 3, 4, 5, 6, 7, 8, 9, or 10 other types of organism, and no other types of organism. The other type of organism in this composition may be, for instance, a bacterium, such as a beneficial bacterium. Suitable beneficial bacteria for this purpose include those in the genera, e.g., Prevotella, Sphingomonas, Pseudomonas, Acinetobacter, Fusobacterium, Megasphaera, Veillonella, Staphylococcus, or Streptococcus. Suitable beneficial bacteria may also include those in the genera, e.g., Bacillus, Lactobacillus, Lactococcus, Streptomyces, Faecalibacterium, Bacteroides, or Bifidobacter.

This disclosure also provides a composition having, e.g., C. accolens and one other type of organism, and no other types of organism. In other examples, the composition has, e.g., C. accolens and 2, 3, 4, 5, 6, 7, 8, 9, or 10 other types of organism, and no other types of organism. The other type of organism in this composition may be, for instance, a bacterium, such as a beneficial bacterium. Suitable beneficial bacteria for this purpose include those in the genera, e.g., Staphylococcus, Corynebacterium, Propionibacterium, Rhodococcus, Microbacterium, or Streptococcus.

Likewise, in some embodiments, a preparation of nonpathogenic bacteria may comprise an effective amount of nonpathogenic bacteria, e.g., ammonia oxidizing microorganisms, sufficient to deliver NO to a subject. In some embodiments, a preparation of nonpathogenic bacteria may comprise an effective amount of ammonia oxidizing microorganisms such that when administered, the preparation modulates, changes, or alters a level of nitrite or NO at a target tissue or in circulation. For instance, a preparation of nonpathogenic bacteria may comprise an effective amount, of ammonia oxidizing microorganisms such that when administered, the preparation results in an increased level of nitrite or NO at a target tissue or in circulation.

The present disclosure provides, inter alia, non-limiting compositions comprising ammonia oxidizing microorganisms, e.g., N. eutropha, e.g., a purified preparation of an optimized N. eutropha. In some embodiments, the N. eutropha in the compositions has at least one property selected from an optimized growth rate, an optimized NH₄⁺ oxidation rate, and an optimized resistance to NH₄⁺.

In some aspects, the present disclosure provides compositions with a defined number of species. A composition may include only one type of species, e.g., one type of nonpathogenic bacteria. This disclosure provides a composition having, e.g., N. eutropha and one other type of organism, and no other types of organism. In other examples, the composition has, e.g., N. eutropha and 2, 3, 4, 5, 6, 7, 8, 9, or 10 other types of organism, and no other types of organism. The other type of organism in this composition may be, for instance, a bacterium, such as an ammonia-oxidizing bacterium. Suitable ammonia-oxidizing microorganisms for this purpose include those in the genera Nitrosomonas, Nitrosococcus, Nitrosospira, Nitrosocystis, Nitrosolobus, or Nitrosovibrio. Likewise, the composition may also include AOA.

A composition may be substantially free of only one type of species, e.g., one type of pathogenic bacteria. This disclosure provides compositions being substantially free of 1, 2, 3, 4, 5, 6, 7, 8, 9, or 10 types of organisms. The types of organisms that may be substantially excluded from the composition may be, for instance, pathogenic bacteria. Suitable pathogenic bacteria for this purpose include, e.g., M. catarrhalis, H. influenzae, S. pneumoniae, S. aureus, V. cholerae, E. coli, or pathogenic bacteria from the species Shigella, Campylobacter, Salmonella, and combinations thereof. A composition may be substantially free of ammonia oxidizing microorganisms, such as ammonia oxidizing bacteria. A composition may be substantially free of N. eutropha.

In some embodiments, the composition comprising nonpathogenic bacteria provides conditions that support nonpathogenic bacterium viability. For instance, the composition may promote growth and metabolism or may promote a dormant state (e.g., freezing, lyophilization, or freeze drying) from which viable nonpathogenic bacteria can be recovered. When the composition promotes growth or metabolism, it may contain water and/or nutrients that nonpathogenic bacteria consume, e.g., as ammonium, ammonia, urea, oxygen, carbon dioxide, or trace minerals. In some embodiments, the composition comprising nonpathogenic bacteria provides conditions that support beneficial bacteria viability. For instance, the composition may promote growth and metabolism or may promote a dormant state (e.g., freezing, lyophilization, or freeze drying) or storage state as described herein, from which viable beneficial bacteria can be recovered. When the composition promotes growth or metabolism, it may contain water and/or nutrients that beneficial bacteria consume, e.g., as ammonium ions, ammonia, urea, oxygen, carbon dioxide, or trace minerals.

In some embodiments, one or more other organisms, for example, organisms besides nonpathogenic bacteria may be included in the preparation of nonpathogenic bacteria. For example, a community of organisms or an organism selected from the group consisting of bacteria, fungi, viruses, and bacteriophages may be administered. In some embodiments, the preparation may be substantially free of other organisms.

Preparations of nonpathogenic bacteria may comprise between about 10³ to about 10¹⁴ CFU/ml. In some embodiments, the preparation of nonpathogenic bacteria may comprise at least about or greater than about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU/ml.

Preparations of nonpathogenic bacteria may comprise between about 10³ to about 10¹⁴ CFU/ml/subject. In some embodiments, the preparation of nonpathogenic bacteria may comprise at least about or greater than about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU/ml/subject.

Preparations of nonpathogenic bacteria may comprise between about 10³ to about 10¹⁴ CFU/ml/m² surface area. In some embodiments, the preparation of nonpathogenic bacteria may comprise at least about or greater than about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU/ml/m² surface area.

Preparations of nonpathogenic bacteria may comprise between about 10³ to about 10¹⁴ CFU/ml/m² floor space. In some embodiments, the preparation of nonpathogenic bacteria may comprise at least about or greater than about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU/ml/m² floor space.

Preparations of nonpathogenic bacteria may comprise between about 10³ to about 10¹⁴ CFU/ml/m³ room volume. In some embodiments, the preparation of nonpathogenic bacteria may comprise at least about or greater than about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU/ml/m³ room volume.

In some embodiments, a preparation of nonpathogenic bacteria may comprise between about 1 × 10⁹ to about 10 × 10⁹ CFU/ml. In some embodiments, an administered dose of the preparation may comprise about 3 × 10¹⁰ CFU, e.g., 3 × 10¹⁰ CFU per day. In some embodiments, an administered dose of the preparation may comprise about 1 × 10⁹ to about 10 × 10⁹ CFU per day, e.g., about 1 × 10⁹ to about 10 × 10⁹ CFU per day. In some embodiments, an administered dose of the preparation may comprise about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per administration or per day.

In some embodiments, an administered dose of the preparation may comprise at least about 7 × 10¹⁰ CFU, e.g., 21 × 10¹⁰ CFU per week. In some embodiments, an administered dose of the preparation may comprise about 1 × 10⁹ to about 10 × 10⁹ CFU per week, e.g., about 1 × 10⁹ to about 10 × 10⁹ CFU per week. In some embodiments, an administered dose of the preparation may comprise about or greater than about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per week.

In some embodiments, an administered dose of the preparation may comprise at least about 30 × 10¹⁰ CFU, e.g., 90 × 10¹⁰ CFU per month. In some embodiments, an administered dose of the preparation may comprise about 1 × 10⁹ to about 10 × 10⁹ CFU per month, e.g., about 1 × 10⁹ to about 10 × 10⁹ CFU per month. In some embodiments, an administered dose of the preparation may comprise about or greater than about 10³, 10⁴, 10⁵, 10⁶, 10⁷, 10⁸, 10⁹, 10¹⁰, 10¹¹, 2 × 10¹¹, 5 × 10¹¹, 10¹², 2 × 10¹², 5 × 10¹², 10¹³, 2 × 10¹³, 5 × 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per month.

In some embodiments, the preparation of nonpathogenic bacteria may comprise between about 0.1 milligrams (mg) and about 1000 mg of nonpathogenic bacteria. In certain aspects, the preparation may comprise between about 50 mg and about 1000 mg of nonpathogenic bacteria. The preparation may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

Advantageously, a formulation may have a pH level that promotes nonpathogenic bacteria viability, e.g., metabolic activity. A formulation may have a pH level that promotes growth or metabolism of nonpathogenic bacteria. A formulation may have a pH that promotes nonpathogenic bacterial colonization of a microbiome of the subject. For example, urea would hydrolyze to ammonia and would raise the pH to 7 to 8. Certain nonpathogenic bacteria, e.g., ammonia oxidizing bacteria are very active at this pH range and would lower the pH to about 6 where the NH₃ converts to ammonium and is unavailable. Lower pH levels, e.g. about pH 4, are also acceptable.

The nonpathogenic bacteria may be combined with one or more pharmaceutically or cosmetically acceptable excipients. In some embodiments, “pharmaceutically acceptable excipient” refers to a pharmaceutically-acceptable material, composition, or vehicle, such as a liquid or solid filler, diluent, solvent, or encapsulating material. In some embodiments, each excipient is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

In some embodiments, a cosmetically acceptable excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

While it is possible for the active ingredient, e.g., nonpathogenic bacteria, to be administered alone, in many embodiments it is present in a pharmaceutical formulation or composition. Accordingly, this disclosure provides a pharmaceutical formulation comprising nonpathogenic bacteria, e.g., beneficial bacteria and a pharmaceutically acceptable excipient. Pharmaceutical compositions may take the form of a pharmaceutical formulation as described below.

In accordance with one or more non-limiting embodiments, a preparation comprising nonpathogenic bacteria may be administered to a subject, e.g., for cosmetic or therapeutic purposes, as a solution, suspension, powder, liquid, drop, spray, aerosol, mist, emulsion, foam, cream, ointment, gel, hydrogel, resin, tablet, capsule, film, suppository, enema, douche, pessary, insert, patch, e.g., transdermal patch, or implantable device, e.g., stent, catheter, vaginal ring, or intrauterine device.

In accordance with one or more embodiments, a preparation of ammonia oxidizing microorganisms may be formulated in order to facilitate a desired delivery mechanism or mode of administration thereof. The formulations, e.g., pharmaceutical or cosmetic formulations, described herein include those suitable for, e.g., oral, enteral (including buccal, sublingual, sublabial, and rectal), parenteral (including subcutaneous, intradermal, intramuscular, intravenous, and intraarticular), inhalation (including fine particle dusts or mists which may be generated by means of various types of metered doses, pressurized aerosols, nebulizers or insufflators, and including intranasally or via the lungs), intranasal, eye, ear, rectal, injection, urogenital, and topical (including dermal, transdermal, transmucosal, buccal, sublingual, and intraocular) administration, although the most suitable route may depend upon, for example, a condition or disorder of a recipient.

Devices configured to deliver a preparation comprising live nonpathogenic bacteria via a desired mode of administration or otherwise via targeted delivery are also disclosed.

In accordance with one or more embodiments, the preparation may be formulated for targeted delivery to a subject, e.g., to a target tissue, region, system, or organ of a subject. For example, the preparation may be formulated for delivery to the eye, ear, nose, urogenital system, respiratory system, or gastrointestinal system of the subject. In some embodiments, targeted delivery may be based on a condition or disorder of a subject. For instance, formulation for targeted delivery may be based on a desired local or systemic effect to be achieved, e.g., a local or systemic therapeutic or cosmetic effect. In some embodiments, a target tissue, region, system, or organ of a subject may be selected for its association with a desired local or systemic effect.

The formulations may conveniently be presented in unit dosage form and may be prepared by any of the methods known in the art of pharmacy. Typically, methods include the step of bringing the active ingredient (e.g., nonpathogenic bacteria) into association with a pharmaceutical carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations may be presented as discrete units such as capsules, cachets or tablets, each containing a predetermined amount of nonpathogenic bacteria; as a powder or granules; as a solution or a suspension in an aqueous liquid or a non-aqueous liquid; or as an oil-in-water liquid emulsion or a water-in-oil liquid emulsion. Formulations, e.g., solutions, aerosols, sprays, and mists, may be presented in multi-dosage form, e.g., packaged units including a predetermined number of dosages, or single dosage form, e.g., packaged units including a single dose. The active ingredient may also be presented as a bolus, electuary or paste. Various pharmaceutically acceptable carriers and their formulation are described in standard formulation treatises, e.g., Remington’s Pharmaceutical Sciences by E. W. Martin. See also Wang, Y. J. and Hanson, M. A., Journal of Parenteral Science and Technology, Technical Report No. 10, Supp. 42:2 S, 1988.

The nonpathogenic bacterial compositions can, for example, be administered in a form suitable for immediate release or extended release. Suitable examples of sustained-release systems include suitable polymeric materials, for example semi-permeable polymer matrices in the form of shaped articles, e.g., films, or microcapsules; suitable hydrophobic materials, for example as an emulsion in an acceptable oil; or ion exchange resins. Sustained-release systems may be administered orally; rectally; parenterally; intracisternally; intravaginally; intraperitoneally; topically, for example as a powder, ointment, gel, drop or transdermal patch; bucally; or as a spray.

Preparations for administration can be suitably formulated to give controlled release of nonpathogenic bacteria. For example, the pharmaceutical compositions may be in the form of particles comprising one or more of biodegradable polymers, polysaccharide jellifying and/or bioadhesive polymers, or amphiphilic polymers. These compositions exhibit certain biocompatibility features which allow a controlled release of an active substance. See U.S. Pat. No. 5,700,486.

Exemplary compositions include suspensions which can contain, for example, microcrystalline cellulose for imparting bulk, alginic acid or sodium alginate as a suspending agent, methylcellulose as a viscosity enhancer, dicalcium phosphate, starch, magnesium stearate and/or lactose and/or other excipients, binders, extenders, disintegrants, diluents and lubricants, mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. The surfactant may be a zwitterionic surfactant, a non-ionic surfactant, or an anionic surfactant.

Excipients, such as surfactants that may be used with embodiments of the present disclosure may include one or more of cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner’s Castile soap, Dr. Bronner’s Castile baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K), and combinations thereof. Dr. Bronner’s Castile soap and Dr. Bronner’s baby soap comprises water, organic coconut oil, potassium hydroxide, organic olive oil, organic fair deal hemp oil, organic jojoba oil, citric acid, and tocopherol.

In some embodiments, surfactants may be used with nonpathogenic bacteria in amounts that allow beneficial byproducts, e.g., nitrite production to occur. In some embodiments, the preparation may have less than about 0.0001% to about 10% of surfactant. In some embodiments, the preparation may have between about 0.1% and about 10% surfactant. In some embodiments, the concentration of surfactant used may be between about 0.0001% and about 10%. In some embodiments, the preparation may be substantially free of surfactant.

In some embodiments, the formulation may include other components that may enhance effectiveness of nonpathogenic bacteria, e.g., beneficial bacteria, delivery thereof, or enhance a treatment or indication.

In some embodiments, a chelator may be included in the preparation. A chelator may be a compound that may bind with another compound, e.g., a metal. The chelator may provide assistance in removing an unwanted compound from an environment, or may act in a protective manner to reduce or eliminate contact of a particular compound with an environment, e.g., nonpathogenic bacteria, e.g. a preparation of nonpathogenic bacteria, e.g., an excipient. In some embodiments, the preparation may be substantially free of chelator.

Formulations may also contain anti-oxidants, buffers, bacteriostats that prevent the growth of undesired microorganisms, solutes, and aqueous and non-aqueous sterile suspensions which may include suspending agents and thickening agents. The formulations may be presented in unit-dose or multi-dose containers, for example sealed ampoules and vials, and may be stored in a freeze-dried or lyophilized condition requiring only the addition of a sterile liquid carrier, for example saline or water-for-injection, immediately prior to use. Extemporaneous solutions and suspensions may be prepared from powders, granules and tablets of the kind previously described. Exemplary compositions include solutions or suspensions which can contain, for example, suitable non-toxic, pharmaceutically acceptable diluents or solvents, such as mannitol, 1,3-butanediol, water, Ringer’s solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition in some embodiments does not include oxidizing agents.

Excipients that can be included are, for instance, proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate. In some embodiments, excipients, e.g., a pharmaceutically acceptable excipient or a cosmetically acceptable excipient, may comprise an anti-adherent, binder, coat, disintegrant, filler, flavor, color, lubricant, glidant, sorbent, preservative, or sweetener. In some embodiments, the preparation may be substantially free of excipients.

In some embodiments, the preparation may be substantially free of one or more of the compounds or substances listed in the disclosure.

Exemplary compositions for spray, aerosol, or mist administration include solutions in saline, which can contain, for example, benzyl alcohol or other suitable preservatives, absorption promoters to enhance bioavailability, and/or other solubilizing or dispersing agents. Conveniently in compositions for aerosol administration the nonpathogenic bacteria is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin can be formulated to contain a powder mix of the nonpathogenic bacteria and a suitable powder base, for example lactose or starch. In certain embodiments, nonpathogenic bacteria are administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations may be presented with carriers such as cocoa butter, synthetic glyceride esters or polyethylene glycol. Such carriers are typically solid at ordinary temperatures, but liquefy and/or dissolve at body temperature to release the nonpathogenic bacteria, e.g., beneficial bacteria.

Exemplary compositions for topical administration include a topical carrier such as Plastibase (mineral oil gelled with polyethylene). In some aspects, the composition and/or excipient may be in the form of one or more of a liquid, a solid, or a gel. For example, liquid suspensions may include, but are not limited to, water, saline, phosphate-buffered saline, or a nonpathogenic bacteria storage buffer. Gel formulations may include, but are not limited to agar, silica, polyacrylic acid (for example Carbopol®), carboxymethyl cellulose, starch, guar gum, alginate or chitosan. In some embodiments, the formulation may be supplemented with an ammonia source including, but not limited to ammonium chloride or ammonium sulfate.

In some embodiments, nonpathogenic bacteria, e.g., beneficial bacteria, composition is formulated to improve beneficial byproduct penetration into the skin or other target tissue. For example, a gel-forming material such as KY jelly or various hair gels would present a diffusion barrier to NO loss to ambient air, and so improve the skin’s absorption of NO. The NO level in the skin will generally not greatly exceed 20 nM/L because that level activates GC and would cause local vasodilatation and oxidative destruction of excess NO.

It should be understood that in addition to the ingredients particularly mentioned above, the formulations as described herein may include other agents conventional in the art having regard to the type of formulation in question.

The formulation, e.g., preparation, e.g., composition may be provided in a container, delivery system, or delivery device, having a weight, including or not including the contents of the container, that may be less than about 50, 100, 200, 300, 400, 500, 600, 700, 800, 900, 1000, 1500, or 2000 grams.

Suitable unit dosage formulations are those containing an effective dose, as hereinbefore recited, or an appropriate fraction thereof, of nonpathogenic bacteria.

A therapeutically effective amount of nonpathogenic bacteria may be administered as a single pulse dose, as a bolus dose, or as pulse doses administered over time. Thus, in pulse doses, a bolus administration of nonpathogenic bacteria is provided, followed by a time period wherein nonpathogenic bacteria is administered to the subject, followed by a second bolus administration. In specific, non-limiting examples, pulse doses are administered during the course of a day, during the course of a week, or during the course of a month.

In some embodiments, a preparation of nonpathogenic bacteria, e.g., a formulation, e.g., a composition, may be applied for a pre-determined number of days. This may be based, for example, at least in part, on the severity of the condition or disease, the response to the treatment, the dosage applied and the frequency of the dose. For example, the preparation may be applied for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days., for about 1 month, for about 2 months, for about 3 months. In some embodiments, the nonpathogenic bacteria is administered for an indefinite period of time, e.g., greater than one year, greater than 5 years, greater than 10 years, greater than 15 years, greater than 30 years, greater than 50 years, greater than 75 years. In certain aspects, the preparation may be applied for about 16 days.

In some embodiments, a preparation of nonpathogenic bacteria, e.g., a formulation, e.g., a composition, may be applied a pre-determined number of times per day. This may be based, for example, at least in part, on the severity of the condition or disease, the response to the treatment, the dosage applied and the frequency of the dose. For example, the preparation may be applied 1, 2, 3, 4, 5, 6, 7, 8, 9, 10, 11, 12, 13, 14, 15, 16, 17, 18, 19, 20, 21, 22, 23, 24 times per day.

In some embodiments, a preparation of nonpathogenic bacteria, e.g., a formulation, e.g., a composition, may be applied for a pre-determined number of minutes or hours, for example, per application. This may be based, for example, at least in part, on the severity of the condition or disease, the response to the treatment, the dosage applied and the frequency of the dose. For example, the preparation may be applied for less than 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 minutes per application. The preparation may be applied for less than about 0.5 hours, 1 hour, 2, 3, 4, 5, 6, 7, 8, 9, 10, 11, 12, 13, 14, 15, 16, 17, 18, 19, 20, 21, 22, 23, or 24 hours per application.

In some embodiments, the preparation may be applied one time per day. In other embodiments, the preparation may be applied two times per day. In some embodiments, the preparation may be applied a first pre-determined amount for a certain number of days, and a second pre-determined amount for a certain subsequent number of days. In some embodiments, the preparation may be applied for about 16 days.

In accordance with one or more embodiments, the preparation may generally be compatible with a physiological environment associated with the subject. In at least some embodiments, compositions are formulated to have a substantially neutral pH or a physiological pH, for instance a pH that normally prevails in the target site for intended delivery, administration, or desired effect. Compositions may be formulated to have a pH between about 5.5 and about 8.5. Compositions may be formulated to comprise compatible conditions, e.g., pH, tonicity, with the target site of physiological environment associated with the subject.

The preparation may be formulated for transmucosal delivery and/or circulation, e.g. locally or systemically. In some embodiments, the preparation may be formulated such that nonpathogenic bacteria, e.g., beneficial bacteria, products thereof, or byproducts thereof (e.g., nitrate, nitrite, NO, or CoQ8) penetrate a deposit or target tissue at least 10%, 20%, 30%, 40%, 50%, 60%, 70%, 80%, 90%, or 100%. The preparation may be formulated such that 10%, 20%, 30%, 40%, 50%, 60%, 70%, 80%, 90%, or 100% of nonpathogenic bacteria, e.g., beneficial bacteria, products thereof, or byproducts thereof, penetrate a deposit or target tissue or enter circulation.

In accordance with one or more embodiments, the preparation may be in the form of a solution, suspension, emulsion, cream, ointment, gel, hydrogel, or liquid, e.g. drop, spray, aerosol, or mist, tablet, capsule, or device for administration to a subject.

In accordance with one or more embodiments, a preparation, composition, formulation, or product comprising nonpathogenic bacteria may undergo quality control and/or testing while it is being made and/or upon its completion. International (PCT) Patent Application Publication No. WO2015/179669 (International (PCT) Patent Application Serial No. PCT/US2015/032017 as filed on May 21, 2015) which is hereby incorporated herein by reference in its entirety for all purposes describes various methods of preparing materials with ammonia oxidizing microorganisms and of testing such materials. For example, one or more parameters such as OD level, pH level, waste level, nutrient level, contaminant level, oxidation rate, nitrite level, protein concentration may be compared against a predetermined value to assess or evaluate an exemplary preparation comprising ammonia oxidizing microorganisms.

The present disclosure provides, inter alia, a kit comprising preparations of nonpathogenic bacteria, as disclosed herein. Formulations may comprise discrete units, e.g., solid, liquid, or gas formulations of nonpathogenic bacteria. Formulations, e.g., solutions, aerosols, sprays, and mists, may be presented in multi-dosage form (multiple use), e.g., packaged units including a predetermined number of dosages, or single dosage form (single use), e.g., packaged units including a single dose. Preparations of nonpathogenic bacteria may be packaged in devices or containers configured to hold a volume of at least less than about 1 ml, 1 ml, 5 ml, 10 ml, 20 ml, 25 ml, 40 ml, 50 ml, 60 ml, 70 ml, 80 ml, 90 ml, 100 ml, or more than about 100 ml.

Kits may further comprise one or more device for administration of the preparation, for example, an inhaler or a nebulizer. The device may comprise a nebulizer and a reservoir. The device, e.g., nebulizer or inhaler, may be mechanically assisted or electrically assisted. In some embodiments, the device, e.g., nebulizer or inhaler, provides a metered dose of the nonpathogenic bacteria. In some embodiments, the device may be configured for multiple use. Kits may comprise a device, for example, syringe, needle, catheter, enema, bulb, pipette (eye or ear dropper), and other devices for drug administration as known in the art. Kits may comprise vials, bottles, pouches, ampoules, and other devices for single-use administration as known in the art. Kits may comprise more than one dose of the preparation comprising nonpathogenic bacteria. Kits may comprise instructions for use, for example instructions for administration of nonpathogenic bacteria as disclosed herein or instructions for combination therapy including administration of nonpathogenic bacteria.

Kits may comprise a second or subsequent composition for administration in conjunction with a nonpathogenic bacterial preparation, as disclosed herein. For instance, kits may comprise a supplement or composition comprising a product or byproduct of nonpathogenic bacteria, a composition that promotes growth or metabolism of nonpathogenic bacteria, a composition that promotes production of products or byproducts of nonpathogenic bacteria, or a composition that has a synergistic effect with nonpathogenic bacteria, or a composition or pharmaceutical agent that treats, e.g., is approved to treat or commonly used to treat, a relevant disease, disorder, or a symptom of a relevant disease or disorder, for example an anti-inflammatory composition. Kits may comprise “biome-friendly” or “biome-compatible” products as disclosed herein, for example one or more microbiome-compatible cosmetic products. Any of the products contained in the kit may be specifically formulated to treat a target indication and/or formulated for a desired mode of delivery, as described herein.

### Natural Products; Consumer Products

In some specific embodiments, a preparation comprising nonpathogenic bacteria as discussed herein may be a natural product or a consumer product. In other embodiments, a preparation of nonpathogenic bacteria may instead be used in conjunction with a natural product or consumer product.

Nonpathogenic bacteria, e.g., beneficial bacteria may be associated with a variety of natural products, and examples of such products are set out below. These natural products may be comprised of formulations, compositions, or preparations disclosed throughout this disclosure.

Natural products may be or comprise products for commercial purposes, and may refer to cosmetics, dietary supplements, and foods, e.g., food, food supplements, medical food, food additive, nutraceutical, or drink, produced from natural sources. Natural products may have pharmacological or biological activity that may be of therapeutic benefit, e.g., in treating disease or conditions. Natural products may be included in traditional medicines, treatments for cosmetological purposes, and spa treatments. A natural product referred to herein may comprise any one or more of the components described as a natural product to be incorporated into a preparation or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

Nonpathogenic bacteria, e.g., beneficial bacteria may be associated with a variety of consumer products, and examples of such products are set out below and be comprised of formulations, compositions, or preparations disclosed throughout this disclosure. In some embodiments, the nonpathogenic bacteria associated with a product is admixed with the product, for example, spread evenly throughout the product, and in some embodiments, nonpathogenic bacteria associated with a product is layered on the product.

In some embodiments, nonpathogenic bacteria are associated with a powder. Powders are typically small particulate solids that are not attached to each other and that can flow freely when tilted. Exemplary powders for consumer use include talcum powder and some cosmetics (e.g., powder foundation).

In some embodiments, the nonpathogenic bacteria are associated with a cosmetic. The cosmetic may be a substance for topical application intended to alter a person’s appearance, e.g., a liquid foundation, a powder foundation, blush, or lipstick, and may be referred to as a preparation. The cosmetic may be any substance recited in the Food and Drug Administration regulations, e.g., under 21 C.F.R.§ 720.4. Other components may be added to these cosmetic preparations as selected by one skilled in the art of cosmetic formulation such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid, or any other excipient known by one of skill in the art, including those disclosed herein.

In some embodiments, the formulations, compositions, or preparations described herein, may comprise, be provided as, or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; a powder (dusting and talcum), a sachet; hair preparations, e.g., hair conditioners, rinses, shampoos, tonics, face powders, cuticle softeners, nail creams and lotions, oral hygiene products, mouthwashes, bath soaps, douches, feminine hygiene deodorants; shaving preparations, e.g., aftershave lotions, skin care preparations, e.g., cleansing, face and neck, body and hand, foot powders and sprays, moisturizing, night preparations, paste masks, skin fresheners; and suntan preparations, e.g., gels, creams, and liquids.

In some embodiments, nonpathogenic bacteria are associated with an aerosol, spray, or mist and these terms may be used interchangeably. An aerosol is typically a colloid of fine solid particles or fine liquid droplets, in a gas such as air. Aerosols may be created by placing the nonpathogenic bacteria (and optionally carriers) in a vessel under pressure, and then opening a valve to release the contents. The container may be designed to only exert levels of pressure that are compatible with nonpathogenic bacterium viability. For instance, the high pressure may be exerted for only a short time, and/or the pressure may be low enough not to impair viability. Examples of consumer uses of aerosols include for sunscreen, deodorant, perfume, hairspray, and insect repellant. The aerosol may be referred to as a spray or mist.

The compositions comprising nonpathogenic bacteria may also comprise one or more of a moisturizing agent, deodorizing agent, scent, colorant, insect repellant, cleansing agent, or UV-blocking agent.

In some embodiments, the product comprising nonpathogenic bacteria is packaged. The packaging may serve to compact the product or protect it from damage, dirt, or degradation. The packaging may comprise, e.g., plastic, paper, cardboard, or wood. In some embodiments the packaging is impermeable to bacteria. In some embodiments, the packaging is permeable to oxygen and/or carbon dioxide.

### Methods of Treatment With Nonpathogenic Bacteria

In accordance with one or more embodiments, a subject may be treated via administration of nonpathogenic bacteria, e.g., a preparation comprising beneficial bacteria. As used herein, treatment of a subject may comprise administering a nonpathogenic bacterial composition for a cosmetic or therapeutic result. For instance, treatment may comprise treating or alleviating a condition, symptom, or side effect associated with a condition or achieving a desired cosmetic effect.

Subjects may include an animal, a mammal, a human, a non-human animal, a livestock animal, research animal, or a companion animal. The subject may be female or male. The subject may have various skin types. The subject may have various health-related profiles, including health history and/or genetic predispositions. The subject may generally have a normal microbiome, e.g., a physiological microbiome, or a disrupted microbiome. The subject may be characterized as one of the following ethnicity/race: Asian, black or African American, Hispanic or Latino, white, or multi-racial. The subject may be of an age of less than 1, or between 1-5, 5-10, 10-20, 20-30, 30-40, 40-50, 50-60, or over 60 years.

The nonpathogenic bacteria that may be used to treat a subject include all the nonpathogenic bacteria, e.g., beneficial bacteria compositions described in this application, e.g. a purified preparation of optimized nonpathogenic bacteria, for instance, an isolated bacterial species or a selected community of species of bacteria. In some embodiments, bacteria that may be used to treat a subject include optimized ammonia oxidizing bacteria, e.g., N. eutropha, e.g., strain D23.

The methods may be provided to administer, or deliver a therapeutic product or a cosmetic product. The methods may comprise administering or introducing a preparation comprising live nonpathogenic bacteria to a subject. The preparation may be formulated to treat a target indication and/or formulated for a desired mode of delivery.

In accordance with one or more embodiments, a preparation comprising live nonpathogenic bacteria may be administered to a first tissue of a subject. The first tissue may be a deposit tissue. The first tissue may be a target tissue or a tissue other than a target tissue. The live nonpathogenic bacteria, or a product thereof, e.g., nitrite and/or nitric oxide, may then move or be transported to a second tissue, e.g., via diffusion. The second tissue may be a target tissue. The target tissue may be associated with a desired local or systemic effect. The target tissue may be associated with an indication, disorder, or condition to be treated.

Nonpathogenic bacterial preparations may be administered, for example to the respiratory system or skin, for a cosmetic or therapeutic effect. For instance, administration may provide a cosmetic treatment, benefit, or effect. In some embodiments, administration may provide for treatment or improvement of one or more of oily appearance, pore appearance, radiance, blotchiness, skin tone evenness, visual smoothness, and tactile smoothness. In some embodiments, a cosmetic appearance of a subject may be altered such as may result from improved skin health. Signs of aging may be reduced, delayed, or reversed. Administration may result in a qualitative improvement in skin and/or scalp condition and/or quality. Skin smoothness, hydration, tightness, and/or softness in a subject may be improved. The present disclosure also provides a method of reducing body odor.

Administration may provide a therapeutic treatment, benefit, or effect. The present disclosure provides a method of modulating or supplying a product or byproduct of beneficial bacteria, e.g., locally or systemically. The present disclosure provides various methods for the suppression, treatment, or prevention of diseases, disorders, infections, and conditions using nonpathogenic bacteria. Nonpathogenic bacteria may be used, for instance, to treat various diseases associated with inflammation or poor endothelial function. In some embodiments, bacteria may be used, for example, to treat diseases associated with suboptimal nitrite levels, skin diseases, and diseases caused by pathogenic bacteria. In some embodiments, administration may provide for a reduction in inflammation. Indeed, a local or systemic anti-inflammatory effect may be demonstrated. In at least some embodiments, microbial growth may be inhibited. Skin and overall health may be improved. Endothelial function may be promoted. A change in level of a product of the nonpathogenic bacteria may be demonstrated. Inadequate circulation may be augmented. For example, a subject’s VO2 max or cardio-metabolic rate may be modulated, e.g., enhanced. In some embodiments, a change in nitrite or NO at a target tissue or in circulation may be demonstrated. In some embodiments, administration, e.g., administration of an effective amount, may modulate, change, or alter a level of nitrite or NO at a target tissue or in circulation. In some embodiments, administration, e.g., administration of an effective amount, may result in a change in the level of nitrite or NO at a target tissue or in circulation.

Administration of the compositions disclosed herein may provide transmucosal delivery and/or circulation, e.g. locally or systemically. In some embodiments, administration may provide that nonpathogenic bacteria, e.g., beneficial bacteria, products thereof, or byproducts thereof (e.g., nitrate, nitrite, NO, or CoQ8) penetrate a deposit or target tissue at least 10%, 20%, 30%, 40%, 50%, 60%, 70%, 80%, 90%, or 100%. In at least some embodiments, 10%, 20%, 30%, 40%, 50%, 60%, 70%, 80%, 90%, or 100% of nonpathogenic bacteria, e.g., beneficial bacteria, products thereof, or byproducts thereof, penetrate a deposit or target tissue or enter circulation upon administration of the compositions disclosed herein.

The preparations and methods of the present disclosure may provide for reducing an amount of undesirable microorganisms, e.g., pathogenic bacteria, from an environment associated with a subject. The nonpathogenic bacteria described herein may out-compete other organisms by, e.g., consuming scarce nutrients, or generating byproducts that are harmful to other organisms, e.g., changing a pH level that is not conducive to the undesirable organism’s growth.

The present disclosure also provides a method of promoting wound healing, including of chronic wounds, such as in a patient that has an impaired healing ability, e.g., a diabetic patient. A bandage including nonpathogenic bacteria, e.g., beneficial bacteria may optionally be applied to the wound.

The present disclosure provides a method of treating respiratory injuries, including, for example, asthma, allergy, carbon monoxide poisoning, smoke inhalation, emphysema, asbestos poisoning, bronchitis, pulmonary fibrosis, cystic fibrosis, embolism, Chronic Obstructive Pulmonary Disease (COPD), adult respiratory distress syndrome, pulmonary hypertension, Celiac’s disease, or pneumonitis. The nonpathogenic bacteria may be administered, for example, via nebulization or inhalation.

It is appreciated that many modern degenerative diseases may be caused by colonization of pathogenic bacteria. Nonpathogenic bacteria, e.g., beneficial bacteria, may be administered directly to a target tissue or via diffusion to a target tissue, to inhibit growth or reproduction of pathogenic bacteria. In some embodiments, a community of nonpathogenic bacteria is selectively administered to a specific target tissue. For example, beneficial bacteria comprising or consisting of the genera including one or more of, e.g., Prevotella, Sphingomonas, Pseudomonas, Acinetobacter, Fusobacterium, Megasphaera, Veillonella, Staphylococcus, or Streptococcus may be administered to a target tissue in the respiratory system. In some embodiments, beneficial bacteria comprising or consisting of the genera including one or more of, e.g., Staphylococcus, Corynebacterium, Propionibacterium, Rhodococcus, Microbacterium, or Streptococcus may be administered to a target tissue in the nasal cavity. Additionally or alternatively, beneficial bacteria comprising or consisting of the genera including one or more of e.g., Bacillus, Lactobacillus, Lactococcus, Streptomyces, Faecalibacterium, Bacteroides, or Bifidobacter may be administered.

It is appreciated that many modern degenerative diseases may be caused by a lack of excipients produced by non-pathogenic bacteria, e.g., NO species. Nonpathogenic bacteria may be administered to supply those species, directly to a target tissue or via diffusion to a target tissue. Ammonia oxidizing microorganisms may be administered to supply NO to a target tissue directly or via diffusion. Application of nonpathogenic bacteria may resolve long standing medical conditions. In certain embodiments, ammonia oxidizing microorganisms are applied to a subject to offset modern bathing practices, especially with anionic detergents which remove ammonia oxidizing microorganisms from the external skin.

In accordance with one or more embodiments, ammonia oxidizing microorganisms convert ammonia to nitrite, an anti-microbial compound, and nitric oxide, a well-documented signaling molecule in the inflammatory process.

The present disclosure provides, inter alia, a method of modulating a composition of a microbiome, e.g., modulating or changing the proportions of a microbiome in an environment, e.g., a surface, e.g., a surface of a subject. This may, in turn, exhibit a health-related benefit. The method may comprise administering a preparation comprising nonpathogenic bacteria to a subject. In some embodiments, the amount and frequency of administration, e.g., application, may be sufficient to reduce a proportion of pathogenic microorganisms.

Application of nonpathogenic bacteria to a subject, e.g., a human subject may lead to unexpected changes in the microbiome. It may lead to increases in the proportion of normal commensal non-pathogenic species and reductions in the proportion of potentially pathogenic, pathogenic, or disease causing organisms.

An increase in the proportion of nonpathogenic bacteria may occur with a pre-determined period of time, e.g., in less than 1 hour, 2 hours, 6 hours, 8 hours, 12 hours, 18 hours, 1 day, 2 days, 3 days, 4 days, 5 days, 1 week, 2 weeks, 3 weeks, or 4 weeks, or in less than 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days.

A decrease in the proportion of pathogenic bacteria may occur with a pre-determined period of time, e.g., in less than 1 hour, 2 hours, 6 hours, 8 hours, 12 hours, 18 hours, 1 day, 2 days, 3 days, 4 days, 5 days, 1 week, 2 weeks, 3 weeks, or 4 weeks, or in less than 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days.

In accordance with one or more embodiments, a subject may be evaluated for need of treatment. In some embodiments, a subject may be selected on the basis of the subject being in need of a treatment. The present disclosure may further provide obtaining a sample from a subject and analyzing the sample. In some embodiments, subjects may be evaluated before, during, and/or after treatment, such as at predetermined time intervals.

In accordance with one or more embodiments, administration may be performed before, during, or subsequent to occurrence of a health-related condition, or in response to a warning sign, trigger, or symptom thereof. In accordance with one or more embodiments, a second amount of the preparation may be administered to the subject, e.g., a second dose.

In certain aspects, the present disclosure provides combination therapies comprising nonpathogenic bacteria and a second treatment, e.g. a therapeutic. For instance, the disclosure provides physical admixtures of the two (or more) therapies are physically admixed. In other embodiments, the two (or more) therapies are administered in combination as separate formulation. The second therapy may be, e.g., a pharmaceutical agent, surgery, diagnostic, or any other medical approach that treats, e.g., is approved to treat or commonly used to treat, the relevant disease, disorder, or a symptom of the relevant disease or disorder. The second treatment may be administered before or after the administration. The effective amount can be administered concurrently with the second treatment. The second treatment may be administered via the same or a different mode of delivery. The subject may have a therapeutic level of the second treatment upon administration of the preparation. In certain embodiments, the second treatment may provide an anti-inflammatory effect or be administered to reduce inflammation at the target site. In at least some embodiments, the preparation may be administered concurrently or in conjunction with a product or byproduct of the nonpathogenic bacteria, e.g., beneficial bacteria. The preparation may be administered concurrently or in conjunction with, e.g., nitrite, nitrate, nitric oxide, CoQ8. In at least some embodiments, the preparation may be administered concurrently or in conjunction with a composition that promotes growth or metabolism of nonpathogenic bacteria, promotes production of products or byproducts of nonpathogenic bacteria, promotes beneficial activity, or has a synergistic effect with nonpathogenic bacteria. The preparation may be administered concurrently or in conjunction with, e.g., ammonia, ammonium salts, urea, and urease.

The preparation may be administered with a microbiome cleansing preparation, for example a local or systemic antibiotic. The preparation may be administered after administration of a broad spectrum antibiotic to “plow the field.” The preparation may be administered after administration of a cleansing preparation or a bowel cleanse. The preparations may be administered pre- or post-surgical procedure, diagnostic procedure, or natural event, e.g., giving birth. The preparations may be administered before, during, or after deposit of an implantable or invasive device, e.g., endotracheal device.

In accordance with one or more embodiments, the preparation may be administered as an analgesic or prophylactic. The preparation may be self-administered. The administration of the preparation may be device-assisted.

In some embodiments, the nonpathogenic bacteria, e.g., a preparation of nonpathogenic bacteria, are administered at a dose of about or greater than about 10³ - 10⁴ CFU, 10⁴ - 10⁵ CFU, 10⁵ - 10⁶ CFU, 10⁶ - 10⁷ CFU, 10⁷ - 10⁸ CFU, 10⁸ - 10⁹ CFU, 10⁹ - 10¹⁰ CFU, 10¹⁰ - 10¹¹ CFU, 10¹¹-10¹² CFU, 10¹²-10¹³ CFU, or 10¹³-10¹⁴ CFU per application, per day, per week, or per month. In some embodiments, the nonpathogenic bacteria are administered at a dose of about 10⁹-10¹⁰ CFU, e.g., about 1 × 10⁹ - 5 × 10⁹, 1 × 10⁹ - 3 × 10⁹, or 1 × 10⁹ - 10 × 10⁹ CFU per application or per day.

In some embodiments, the nonpathogenic bacteria are administered in a volume of about 1-2, 2-5, 5-10, 10-15, 12-18, 15-20, 20-25, or 25-50 ml per dose. In some embodiments, the solution is at a concentration of about 10⁸-10⁹, 10⁹-10¹⁰, or 10¹⁰-10¹¹ CFU/ml. In some embodiments, the nonpathogenic bacteria are administered as two doses per day, where each dose is at a concentration of 10⁹ CFU/ml.

In some embodiments, the nonpathogenic bacteria are administered once, twice, three, or four times per day. In some embodiments, the nonpathogenic bacteria are administered once, twice, three, four, five, or six times per week. In some embodiments, the nonpathogenic bacteria are administered shortly after bathing. In some embodiments, the nonpathogenic bacteria are administered shortly before bathing. In some embodiments, the nonpathogenic bacteria are administered shortly before sleep. In some embodiments, the nonpathogenic bacteria are administered shortly after sleep.

In some embodiments, the nonpathogenic bacteria are administered for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, e.g., for about 1 month, for about 2 months, for about 3 months. In some embodiments, the nonpathogenic bacteria are administered for an indefinite period of time, e.g., greater than one year, greater than 5 years, greater than 10 years, greater than 15 years, greater than 30 years, greater than 50 years, greater than 75 years.

### Administration of Nonpathogenic Bacteria to an Environment

Microorganism preparations may be used in an environment to provide a health benefit. In accordance with certain aspects, the preparations may be used to alter a microbiome of an environment. The preparations may be used to prevent, limit, or reduce the spread of pathogenic bacteria within the environment, for example, to a subject in the environment. In some embodiments, the preparations may be used for infections disease control.

Microorganism preparations may be administered, e.g., applied, to a built environment. As disclosed herein, a built environment may include any defined space. The defined space may be part of a built structure. The built environment may be a substantially enclosed space. The preparation may be administered to a defined space within the built environment, e.g., a room. The substantially enclosed space may include a building, dwelling, or substantially enclosed mode of transportation (e.g., car, airplane, train, boat). The substantially enclosed space may be a subset of the building, dwelling, or mode of transportation, e.g., a room, a defined plurality of rooms, or any other section protected by a separator. The preparations may be administered substantially continuously or periodically. The building may be commercial or residential. In some embodiments, the building may include a hospital or health care facility, an apartment building, a hotel, a retail store, a shopping center or mall, a school or vocational center, an educational center or cultural institution, an office building, an indoor park, gymnasium or recreational building, or a transportation hub, e.g., airport, train station, bus station, or subway station.

Microorganism preparations may be administered, e.g., applied, to an environmental surface. The environmental surface may be permanently fixed or portable. The environmental surface may be associated with an inanimate object, a surface of a building, a construction material, an assembly material or component, an article of furniture or other portable object, or a fixture.

The formulations (e.g., preparations or compositions) described herein may include those suitable for delivery to a built environment or environmental surface via auxiliary system or appliance. Nonpathogenic bacterial formulations may be delivered to a subject, e.g., skin, scalp, gastrointestinal system, or respiratory system of the subject. Nonpathogenic bacterial formulations may be alternately or additionally delivered to a subject orally, intranasally, enterally, topically, ocularly, via the auditory system, via the urogenital system. Inhaled nonpathogenic bacterial formulations may be delivered to the respiratory system via the auxiliary system or appliance. Nonpathogenic bacteria may be introduced to a subject via contact with the environmental surface or built environment for cosmetic or therapeutic purposes. For instance, compositions include those formulated for cosmetic or therapeutic use.

The nonpathogenic bacterial formulations (e.g., preparations or compositions) may conveniently be presented in unit dosage form and may be prepared by any of the methods known in the art of building design, consumer product design, pharmacy, or cosmetology. Typically, methods include the step of bringing the active ingredient (e.g., nonpathogenic bacteria) into association with a carrier, e.g., liquid, gas, or pharmaceutical carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Bacterial formulations may be presented as discrete units, each containing a predetermined amount of the active ingredient as a solution or suspension in an aqueous or nonaqueous liquid, as a powder or granules, or as an oil-in-water or water-in-oil liquid emulsion. Various exemplary pharmaceutically acceptable carriers and their formulation are described in standard formulation treatises, e.g., Remington’s Pharmaceutical Sciences by E.W. Martin. See also Wang, Y.J. and Hanson, M.A., Journal of Parenteral Science and Technology, Technical Report No. 10, Supp. 42:2 S, 1988; Aulton, M. and Taylor, K., Aulton’s Pharmaceutics: The Design and Manufacture of Medicines, 5^{th} Edition, 2017; Antoine, A., Gupta M.R., and Stagner, W.C., Integrated Pharmaceutics: Applied Preformulation, Product Design, and Regulatory Science, 2013.

Compositions disclosed herein may be formulated for device-assisted application with an auxiliary system, for example, a system for controlled environment, for example, a heating, ventilation, and air conditioning (HVAC) system. Such compositions may be prepared in HVAC dosage formulations. Nonpathogenic bacteria can be introduced to a built environment via the auxiliary system. Nonpathogenic bacteria can be introduced to a subject via the auxiliary system for cosmetic or therapeutic purposes. For instance, compositions may be prepared as solutions (e.g., ultrafine droplets). The solutions may be formulated to comprise one or more carrier or excipient, as described in more detail below. Typically, such solutions, e.g., for administration via HVAC system, may be aqueous solutions, e.g., an aqueous dispersion or suspension of the active agent. Solutions may be formulated as a colloidal suspension of particles or droplets dispersed in air or gas. The solutions may be in the form of a mist, for example, a dispersion of small liquid droplets suspended in a gas. The solutions may be in the form of humidity, for example, a dispersion of ultrafine liquid particles suspended in a gas. Alternatively, the preparation may be formulated as a dry powder suspended in a gas carrier to be distributed via the auxiliary system.

Compositions disclosed herein may be formulated for device-assisted application with an appliance, e.g., a humidifier or air conditioner. The humidifier may be a central humidifier, an evaporator, an impeller humidifier, a steam vaporizer, cool mist humidifier, or an ultrasonic humidifier. The humidifier may operate by a combination of methods, e.g., ultrasonic and impeller humidifying. In general, the humidifier may refrain from heating the preparation directly to a temperature greater than between about 55° C. and about 60° C. The humidifier may operate by bubbling evaporated water, heated water, or steam through the preparation without directly heating the preparation. The air conditioner may include a mini-split air conditioner, window air conditioner, portable air conditioner, hybrid air conditioner, or geothermal air conditioner. As disclosed herein, the air conditioner may provide cooling, heating, or both. The air conditioner may optionally be operated in a “fan” setting, without providing cooling or heating.

Compositions disclosed herein may be formulated for device-assisted application with an appliance, e.g., a dehumidifier or air purifier. The dehumidifier may be a refrigerator coil dehumidifier or desiccant dehumidifier. The air purifier may include a HEPA filter, ionizer, ozone generator, electrostatic filter, activated carbon filter, or ultraviolet filter. In certain embodiments, the preparation may be introduced downstream from the air purifying component of the air purifier, so as to ensure viability of the microorganisms.

Nonpathogenic bacteria can be introduced to a built environment via the appliance. In some embodiments, nonpathogenic bacterial formulations may be introduced to a subject via the appliance for cosmetic or therapeutic purposes. Such formulations may be prepared as solutions or dry powders, e.g., in a single use cartridge. Compositions may be prepared as dry powders, e.g., lyophilized particles. Dry powder formulations may comprise particles, e.g., lyophilized particles, in a gas medium or carrier. Such formulations for administration via appliance may be a dry powder suspended in a gas carrier. Formulations may also be prepared as solutions, e.g., ultrafine droplets. Formulations for administration via appliance may comprise one or more carrier or excipient. Aqueous solutions may be formulated as a colloidal suspension of particles or droplets dispersed in air or gas. Solutions may be in the form of a mist, for example, a dispersion of small liquid droplets suspended in a gas carrier. The solutions may be in the form of humidity, for example, a dispersion of ultrafine liquid particles suspended in a gas.

The auxiliary system or appliance may distribute the formulation by propulsion. The formulation may be provided as a cartridge to be installed in the auxiliary system or appliance. For example, the formulation in dry powder or aqueous format may be contained within a cartridge. The auxiliary system or appliance may include a chamber or reservoir for the formulation, in dry powder or aqueous format. The auxiliary system or appliance may refrain from providing direct heat to the formulation. For example, the auxiliary system or appliance may refrain from heating the preparation directly to a temperature greater than between about 55° C. and about 60° C. In some embodiments, the auxiliary system or appliance may provide cooling to the formulation. For example, the chamber or reservoir can be refrigerated or otherwise maintained at a temperature of between about 2° C. and about 10° C., for example, about 4° C.

A preparation of nonpathogenic bacteria may be administered as a plurality of particles having a predetermined particle size range (PSR), e.g., as droplets or dry particles. A mixture comprising nonpathogenic bacteria may be subject to conditions that form a plurality of particles of the mixture having the predetermined PSR. The mixture may comprise, consist essentially of, or consist of nonpathogenic bacteria. In some embodiments, the mixture may further comprise a carrier, e.g., a liquid or gas carrier. A volume of the mixture may be provided, for example, in a device capable of forming the mixture into particles. In some embodiments, the volume may be separated from the mixture, for example, to subject the volume to conditions that form the plurality of particles.

Nonpathogenic bacterial formulations for administration to a subject may comprise particles, e.g., droplets or powders, in a liquid or gas medium carrier. In some embodiments, particles may have a PSR of about 5 µm or less, about 10 µm or less, about 25 µm or less, about 50 µm or less, about 100 µm or less, about 250 µm or less, about 400 µm or less, or about 500 µm or less. Generally, compositions for delivery to a built environment or environmental surface may be formulated to have a PSR in the range of about less than 1 µm to about 10 µm, for example, having a PSR between about 0.1 µm and about 1 µm or between about 0.1 µm and about 5 µm. Compositions for delivery to a built environment or environmental surface may have an PSR in the range of about 10 µm or greater, for example, between about 10 µm and about 25 µm, between about 10 µm and about 50 µm, or between about 10 µm and about 100 µm. In some embodiments, compositions may have a PSR between about 1 µm and about 50 µm, between about 1 µm and about 100 µm, or between about 5 µm and about 50 µm.

In some embodiments, at least about 1%, 5%, 10%, 20%, 30%, 40%, 50%, 60%, 70%, 80%, 85%, 90%, 95%, or 99% of the volume is converted to particles in the PSR. At least 99%, 99.9%, 99.99%, or 99.999% of the volume may be converted to particles in the PSR. In some embodiments, substantially all of the volume is converted to particles in the PSR, e.g., particles having a PSR of less than 1 µm to about 5 µm, e.g., between about 0.1 µm and about 5 µm.

Compositions formulated for delivery to a built environment or environmental surface may generally comprise a water-soluble or highly water-insoluble formulation. Water, oil, or buffer may be used as a solvent for the formulation. Generally, such formulations may have compatible conditions with a deposit or target tissue, for example a substantially compatible pH, tonicity, and/or surface tension. The formulations may further comprise a tonicity-adjusting agent to ensure proper tonicity for the solution, a buffer to maintain stability and solubility of the solution, a surfactant, and/or an antioxidant. Generally, the formulations may be isotonic and have a relatively neutral pH. In some embodiments, the formulations have a pH of between about 5.5 and 8.5, for example, between about 6 and 8, for example, about 7.

The bacterial formulations can, for example, be administered in a form suitable for immediate release or extended release. Immediate release formulations may comprise penetration enhancers, for example, sugars and cyclodextrins. Immediate release formulations may comprise enzyme inhibitors. Preparations for administration can also be suitably formulated to give controlled release of nonpathogenic bacteria. In some embodiments, controlled release formulations may comprise polymer carriers. However, the safety of the polymer carriers in the respiratory system is of great concern. Dosage formulations for controlled release may comprise polymer carriers that are compatible with lung fluid and do not accumulate significantly within the lung.

In some embodiments, bacterial formulations comprising aqueous solutions may provide onset action within minutes of administration. For example, formulations comprising aqueous solutions may provide onset action within about 60 minutes of administration, within about 45 minutes of administration, within about 30 minutes of administration, or within about 15 minutes of administration.

Bacterial formulations disclosed herein may comprise an effective amount of nonpathogenic bacteria, for example, to penetrate a target tissue, to colonize a tissue, e.g., a target tissue of the lung or nasal cavity, to treat a disorder or a symptom of a disorder, or to promote endothelial function, e.g., within the gastrointestinal system, respiratory system, or topically. Bacterial formulations may comprise an effective amount to provide a health benefit or otherwise support a healthy lifestyle. The preparation may be formulated to provide a health benefit to at least one of the skin, scalp, gastrointestinal system, intranasal cavities, and respiratory system. Bacterial formulations may comprise an effective amount of nonpathogenic bacteria to treat a local or systemic disorder or a symptom of a local or systemic disorder.

In some embodiments, bacterial formulations may comprise an effective amount of nonpathogenic bacteria to colonize or inoculate a target tissue, e.g., skin, the scalp, or a tissue of the respiratory system. Bacterial formulations may comprise an effective amount of nonpathogenic bacteria to supplement or modulate a microbiome of a subject. The microbiome of the subject may be, for example, a skin microbiome, an oral microbiome, an enteral microbiome, an optical microbiome, an auditory microbiome, a lung microbiome, nasal microbiome, or others. A mixture comprising nonpathogenic bacteria may be prepared from a nonpathogenic bacterial sample. Nonpathogenic bacterial formulations may comprise a monoculture of organisms, e.g., may be selected for a specific microbiological organism, or a community of organisms, e.g., may be selected for a specific community of organisms. The nonpathogenic bacterial preparation may be prepared in vitro. Generally, nonpathogenic bacteria may include non-harmful and non-virulent bacteria. Nonpathogenic bacteria may include beneficial bacteria, for example, bacteria associated with a subject’s microbiome or which provide a benefit to a subject’s microbiome.

Nonpathogenic bacteria may include bacteria selected from any one or more of the following genera: Prevotella, Phingomonas, Pseudomonas, Acinetobacter, Fusobacterium, Megasphaera, Veillonella, Staphylococcus, or Streptococcus. Nonpathogenic bacteria may include bacteria selected from any one or more of the following genera: Staphylococcus, Corynebacterium, Propionibacterium, Rhodococcus, Microbacterium, or Streptococcus. Nonpathogenic bacteria may include bacteria selected from any one or more of the following genera: Bacillus, Lactobacillus, Lactococcus, Streptomyces, Faecalibacterium, Bacteroides, or Bifidobacter. The nonpathogenic bacteria may comprise lactic-acid producing microorganisms, for example, probiotics. The nonpathogenic bacteria may comprise ammonia oxidizing bacteria (AOB). The nonpathogenic bacteria may comprise Nitrosimonas, Nitrosococcus, Nitrosospira, Nitrosocystis, Nitrosolobus, or Nitrosovibrio. The nonpathogenic bacteria may comprise Nitrosimonas eutropha (N. eutropha), for example, N. eutropha D23 (having ATCC accession number PTA-121157). The nonpathogenic bacteria may be substantially free of AOB. The nonpathogenic bacteria may be substantially free of pathogenic or potentially pathogenic bacteria, e.g., M. catarrhalis, H. influenzae, S. pneumoniae, S. aureus, V. cholerae, E. coli, species from the genera including Shigella, Campylobacter, or Salmonella, and combinations thereof. The nonpathogenic bacteria may comprise at least 10%, 20%, 30%, 40%, 50%, 60%, 70%, 80%, 85%, 90%, 95%, or 99% live bacteria. The nonpathogenic bacteria may comprise at least 99%, 99.9%, 99.99%, or 99.999% live bacteria. In some embodiments, substantially all of the nonpathogenic bacteria may be live bacteria.

The preparation comprising nonpathogenic bacteria may be prepared in vitro, for example, from a bacterial sample. The sample may be purified to comprise a desired concentration of nonpathogenic bacteria. The sample may be kept in a dormant state or may be induced to a reproductive state. In some embodiments, the sample may be cultured for at least a desired number of cell divisions, e.g., until the bacteria have reproduced for at least 1, at least 2, at least 5, or at least 10 generations. The sample may be cultured for no more than a desired number of cell divisions e.g., before the bacteria have reproduced for at most 1, at most 2, at most 5, or at most 10 generations. The time of culture for the desired number of cell divisions will depend on the species of bacteria in the sample. The sample may be cultured until the bacteria reach a stationary state, for example, due to exhaustion of nutrients, accumulation of inhibitory metabolites or end products, or exhaustion of biological space. The sample may be purified or processed to comprise at least 10%, 20%, 30%, 40%, 50%, 60%, 70%, 80%, 85%, 90%, 95%, or 99% nonpathogenic bacteria. The sample may be purified or processed to comprise at least 99%, 99.9%, 99.99%, or 99.999% nonpathogenic bacteria. The sample may be purified to consist or consist essentially of nonpathogenic bacteria. The preparation may be purified to contain an isolated bacterial species or to be selective for a community of specific nonpathogenic bacteria. In some embodiments, the sample may be passaged, e.g., more than once, to be selective for a specific species or community of nonpathogenic bacteria.

In some embodiments, the bacterial sample may be derived from a biological sample. The biological sample may comprise nonpathogenic bacteria which are present or can typically be found in the microbiome of a desired class of subjects. For instance, the nonpathogenic bacteria may be present or found in a mammalian, e.g., human microbiome. The nonpathogenic bacteria may be present or found in a specific microbiome, e.g., a gastrointestinal, respiratory, or skin microbiome. In some embodiments, the nonpathogenic bacteria may be present or found in a mouth, gut, colon, fecal, nasal cavity, trachea, lung, or skin microbiome. In certain embodiments, the nonpathogenic bacteria may be present or typically found in the microbiome of a healthy or non-diseased mammal, e.g., human.

The nonpathogenic bacteria may be derived from a donor. In some embodiments, the method may comprise collecting a biological sample from a donor. The donor may be selected or evaluated for acceptability, e.g., for meeting a predetermined criteria or reference. The donor may generally be evaluated for meeting a health requirement. The donor may be healthy, e.g., physiological, normal, or non-diseased. The donor may have a healthy microbiome, e.g., physiological, normal, or non-diseased microbiome. The donor may be evaluated for presence or absence of a condition or disorder, e.g., a respiratory or gastrointestinal condition or disorder. The donor may generally be free of symptoms which indicate presence of a condition or disorder, e.g., a respiratory or gastrointestinal condition or disorder. A donor may be selected or rejected responsive to the evaluation. A biological sample may be obtained from the donor responsive to the evaluation, for example, if the evaluation meets the predetermined criteria.

The donor’s microbiome may comprise a desired concentration of nonpathogenic bacteria or beneficial bacteria. The biological sample, e.g., microbiome of the biological sample, may be selected or evaluated for acceptability, e.g., for meeting a predetermined criteria or reference. For example, the biological sample may be analyzed for bacterial composition prior to transfer to a recipient. The biological sample may have a healthy microbiome, e.g., physiological, normal, or non-diseased microbiome. The biological sample may be evaluated for a threshold concentration of pathogenic bacteria. The biological sample may be selected or rejected responsive to the evaluation. For example, the biological sample may be selected if the evaluation meets the predetermined criteria. In some embodiments, the biological sample is selected if it is determined to comprise less than the threshold concentration or be free or substantially free of pathogenic bacteria, e.g., M. catarrhalis, H. influenzae, S. pneumoniae, and S. aureus. The biological sample may be selected if it is determined to comprise less than a threshold concentration of, e.g., V. cholerae, E. coli, or species from the genera Shigella, Campylobacter, or Salmonella. In some embodiments, the biological sample is transferred to a recipient before the cells undergo any significant number of cell divisions, for example, while they are held in a dormant state. The biological sample may be collected and isolated until it is to be transferred to a subject. The biological sample may be collected and cultured for a desired number of cell divisions, as described above. The subject, e.g., recipient, may have a disrupted microbiome. For example, the microbiome of the subject, e.g., gastrointestinal, respiratory, or skin microbiome, may comprise pathogenic bacteria or be diseased. The microbiome of the subject, e.g., gastrointestinal, respiratory, or skin microbiome, may comprise more than a threshold concentration of, e.g., M. catarrhalis, H. influenzae, S. pneumoniae, S. aureus, V. cholerae, E. coli, or species from the genera Shigella, Campylobacter, Salmonella, or combinations thereof.

The preparations may be administered to a healthy subject. In accordance with certain embodiments, the preparations may be administered to a non-diseased subject. The subject may be healthy, e.g., physiological, normal, or non-diseased. The subject may have a healthy microbiome, e.g., physiological, normal, or non-diseased microbiome. The preparations may be administered in an effective amount to provide a health benefit or support a healthy lifestyle. The preparations may be administered in an effective amount to module or maintain a microbiome of a subject, e.g., a healthy subject, e.g., physiological, normal, or non-diseased subject. In accordance with certain embodiments, the preparations may be administered to a subject who is not exhibiting signs or symptoms of a disease requiring treatment or for which treatment is recommended.

The preparations may be administered to a subject in need of treatment. The bacterial compositions can, for example, be administered in form suitable to provide local therapeutic treatment or systemic therapeutic treatment. Compositions disclosed herein may be administered to treat a local inflammatory disease, a symptom of a local or systemic inflammatory disease, or a side effect caused by a local or systemic inflammatory disease. Suitable examples of local respiratory conditions that may be treated with compositions disclosed herein include infection, inflammation, and congestion of the trachea, lungs, or a respiratory system tissue. Local conditions or disorders associated with the respiratory system may include airway diseases, cardiac disorders, vascular disorders, or pulmonary disorders. For instance, local conditions or respiratory disorders may include asthma, allergy, carbon monoxide poisoning, smoke inhalation, bronchitis, pulmonary fibrosis, cystic fibrosis, embolism, adult respiratory distress syndrome, pulmonary hypertension, pneumonitis, Chronic Obstructive Pulmonary Disease (COPD), and Celiac’s disease. In some embodiments, the respiratory disorder may be associated with or comprise an airway disorder, a cardiac disorder, a vascular disorder, or a pulmonary disorder. In some embodiments, bacterial compositions can be administered in a form suitable to treat certain infections and inflammatory disorders, e.g., a respiratory state of inflammation, bacterial infections, fungal infections, viral infections, itching, local inflammation, and wound healing. For instance, bacterial compositions may be administered to treat inflammation associated with a surgical or diagnostic procedure, catheter-based transfers (e.g., matter transfers in, out, or in between two locations within the body), endotracheal intubation, stents, or generally inflammation related to any foreign body introduced into the respiratory system. Bacterial formulations may be administered to treat localized symptoms of respiratory conditions, disorders, or systemic disorders, e.g., coughing, difficulty breathing, congestion, inflammation, infection of the lungs, trachea, or other respiratory system tissue, or side effects associated with such conditions, disorders, or systemic disorders, e.g., asthma, allergy, poisoning, bronchitis, or pneumonitis. In at least some embodiments, administration of bacterial compositions may reduce a symptom or side effect associated with a respiratory condition or disorder, e.g., congestion, inflammation, or infection.

Suitable examples of local skin conditions that may be treated with compositions disclosed herein, e.g., when applied topically via nebulization, include skin disorders such as hyperhidrosis, pruritus, contact dermatitis, atopic dermatitis, skin infections, soft tissue infections, and impetigo. Wounds associated with, e.g., bed sores, burns, and diabetic ulcers, symptoms and side effects of wound healing, e.g., biofilm, and infected wounds or chronic wounds can be treated with the nonpathogenic bacterial formulations disclosed herein. Local topical inflammatory conditions that may be treated include, e.g., seborrheic dermatitis, acne, acne vulgaris, inflammatory lesions, papules, pustules, cysts, nodules, comedones (open or closed), post-inflammatory hyperpigmentation, post-inflammatory erythema, erythema, edema, scaling, stinging, burning, lichen planus, eczema, hives, idiopathic uriticaria, uriticaria, insect bites, poison ivy, itch, keratosis pilaris, pemphigus, psoriasis, rosacea, folliculitis, hidradinitis suppurativa, dermatomyositis, and lupus rash. Topical application of the compositions disclosed herein may protection against, e.g., sunburns, mosquito and bug bites, and demodex folliculum mite bites. Compositions disclosed herein may be applied topically as a bug or pest repellant. Fungal infections that may be treated by, e.g., topical application, of the compositions disclosed herein include dandruff, tinea cruris, tinea pedis/fungus, tinea unguium, onychomycosis (toe fungus), tinea versicolor, and athelete’s foot. Bacterial infections that may be treated by, e.g., topical application, of the compositions disclosed herein include MRSA/Staph and infections caused by one or more of, e.g., Pseudomonas aeruginosa, Streptococcus pyogenes, Acinetobacter baumanii, Propionibacteria, Stenotrophomonas, and Proponibacterium acnes. Nonpathogenic bacterial compositions disclosed herein may be applied, e.g., topically, to treat or improve one or more of body odor, skin appearance, skin blotchiness, skin hydration, sun spots, heloma, oily appearance, sebum secretion, pore appearance, skin radiance, skin tone evenness, visual smoothness, tactile smoothness.

Nonpathogenic bacterial formulations disclosed herein may be administered to increase the amount of oxygen that the subject utilizes during exercise, e.g., intense or maximal exercise. For instance, the formulations may be administered to increase the subject’s VO2 max. A subject’s VO2 max or cardio-metabolic rate may be enhanced during certain activities, for example, during exercise or when the subject is located at a high elevation. The formulations, e.g., nebulized or inhaled formulations, may be delivered via face mask at the high elevation. The formulations may be delivered through or in conjunction with an elevation mask.

Examples of systemic conditions that may be treated with compositions disclosed herein include headaches, cardiovascular diseases, connective tissue disorders, inflammation, immune responses and autoimmune disorders, liver diseases, infections, neurological diseases, psychiatric disorders, nitric oxide disorders, urea cycle disorders, congestion, vasodilation disorders, skin diseases, ophthalmic disorders, wound healing, bowel disorders, reactions to insect bites, and certain viral, bacterial, and fungal infections. For instance, systemic conditions that may be treated with compositions disclosed herein include cardiovascular diseases such as cardioprotection, heart failure, hypertension, pulmonary arterial hypertension; immune responses and autoimmune disorders such as alopecia and vitiligo; liver diseases such as non-alcoholic fatty liver disease (NAFLD), non-alcoholic steatohepatitis (NASH); neurological diseases and psychological disorders such as depression, insomnia, and diabetic neuropathy; nitric oxide disorders such as erectile dysfunction; wound healing, e.g., from bed sores and nursing home care, burns, diabetic ulcers e.g., foot ulcer, venous leg ulcer, biofilm, and mouth sores; skin diseases and disorders such as hyperhydrosis, pruritus, helomas, and subtypes of helomas; ophthalmic disorders such as blepharitis, dry eye, macular degeneration, and glaucoma; bowel disorders such as gluten sensitivity, irritable/inflammatory bowel disease, Crohn’s disease, colitis, and necrotizing enterocolitis; and vasodilation disorders such as Renaud’s disease, thermoregulation, and migraines. Certain viral, bacterial, and fungal infections may be treated with formulations disclosed herein, including infections caused by human papillomavirus (HPV), yeast infections, tinea versicolor, tinea unguium, tinea pedis/fungus, tinea cruris, jock itch, onychomycosis, dandruff, athlete’s foot, sinusitis, otitis media, Methicillin-resistant Staphylococcus aureus (MRSA), staph, and bacterial vaginosis. Additional systemic conditions that may be treated with compositions disclosed herein include systemic inflammation, such as eczema, e.g., adult and pediatric eczema, hives, idiopathic uriticaria, lichen planus, insect bites including allergic reactions to insect bites, e.g., mosquito and demodex folliculorum mite, reactions to poison ivy, itchiness, keratosis pilaris, laryngitis, pemphigus, psoriasis, rosacea, folliculitis and subtypes of folliculitis, hidradenitis supportiva, perioral dermatitis, lupus rash, seborrheic dermatitis, e.g., adult and infantile seborrheic dermatitis, acne, e.g., adolescent acne, adult acne, and cystic acne, diaper rash, occupational hand dermatitis, sunburn, and dermatomyositis. Additionally, compositions disclosed herein may be delivered or applied to treat certain cosmetic indications, including but not limited to, contact dermatitis, diaper odor, e.g., adult and pediatric, body odor, feminine odor, flaking, nail hardness, body odor, oily skin, razor burn, skin appearance, skit blotchiness, skin hydration, and sun spots. Compositions disclosed herein may be applied as a bug repellant or an antimicrobial agent.

Exemplary compositions may further include one or more excipients, for example, absorption and penetration enhancers, preservatives, antioxidants, buffers, chelating agents, ion exchange agents, solubilizing agents, suspending agents, thickeners, surfactants, wetting agents, tonicity-adjusting agents, enzyme inhibitors, and vehicle for proper drug delivery. Absorption and penetration enhancers, e.g., cyclodextrins, may improve the ability of the active agent to be absorbed by a number of different mechanisms. Antioxidants may reduce the oxidative degradation of the active agent. Buffers may maintain a desired pH of the composition and/or enhance solubility or stability of the composition. Chelating agents may include complex trace metals that catalyze oxidation reactions of the composition. Ion exchange agents may control the release of active agent by ion exchange mechanisms. Solubilizing agents may increase the solubility of the active agent or another excipient. Suspending agents and thickeners may increase the viscosity or density of the composition to decrease settling rate of a dispersed material and/or increase the active agent’s retention time and residence time in the target system. Surfactants, including cationic, anionic, and non-ionic surfactants, and wetting agents may act to wet insoluble hydrophobic active agent or other excipients. Tonicity-adjusting agents may provide an isotonic solution with target system tissues. Vehicle, for example water, may provide bulk for proper active agent delivery. Specifically, the vehicle may comprise liquefied inert propellant or a gas inert propellant.

In some non-limiting embodiments, the preparations may be one or more of: substantially odorless, colorless, not associated with substantial side effects, non-toxic, well-tolerated, have no adverse effects if released into the environment, no risk of fostering antibiotic resistance, and have a physiology such that it can interact positively with various human microbiomes, e.g., microbiomes associated with a target tissue, under normal and disease states.

Compositions disclosed herein may further be formulated as combination therapies. Initial and subsequent therapeutic treatments may be provided in a single dosage form, prepared in individual dosage forms, administered concurrently, or administered separately. Individual dosage forms may be administered via the same mode of administration, e.g., to the built environment or to an environmental surface, or to the subject via an alternate mode of administration, e.g., orally, intranasally, enterally, topically, ocularly, via the auditory system, via the urogenital system, via the gastrointestinal system, via injection, or via the respiratory system. For instance, combination therapies may comprise nonpathogenic bacteria for treatment of an inflammatory disease or condition. Individual dosage forms may be administered by a surgical or diagnostic procedure. In some embodiments, bacterial compositions disclosed herein are formulated for combination therapy with an anti-inflammatory. Generally, compositions disclosed herein may be formulated for combination therapy with a drug or compound approved or commonly used to treat a disease, disorder, condition, symptom thereof, or side-effect thereof, for example a respiratory disease, disorder, condition, symptom thereof, or side-effect thereof.

In at least some embodiments, nonpathogenic bacterial therapeutic compositions may be administered in combination with a therapeutic treatment for asthma or Chronic Obstructive Pulmonary Disease (COPD). For instance, combination therapies may comprise a preparation for treatment of congestion, inflammation, infection, or a symptom of a respiratory disease or condition. In some embodiments, bacterial compositions, for example prepared for inhalation via nebulization, are formulated for combination therapy with asthma medication, cold and flu medication, corticosteroids, or anti-histamines.

Preparations for administration to the built environment or environmental surface may be formulated for targeted delivery to a specific deposit tissue or target tissue. In some embodiments, a preparation may be administered to a first tissue such that the preparation or a product of the preparation, e.g., nonpathogenic bacteria or a product of such bacteria, is transported to a second tissue. The first tissue may be a deposit tissue. The second tissue may be a target tissue. The deposit tissue and the target tissue may be the same or different tissues. In some embodiments, the deposit tissue, the target tissue, or both may be a topical tissue, e.g., skin or scalp. In some embodiments, the deposit tissue, the target tissue, or both may be a tissue of the respiratory system. The preparation or product of the preparation may be delivered locally or systemically, e.g., at a deposit or target tissue or in circulation.

The respiratory deposit or target tissue may be associated with a nose, nasopharynx, larynx, or trachea of a subject. The respiratory deposit or target tissue may comprise or be associated with a mucous membrane, alveolar epithelium, or pulmonary parenchymal tissue of the subject. Specifically, respiratory deposit or target tissues may include the lung, trachea (wind pipe), larynx, pharynx, bronchioles, segmental bronchi, subsegmental bronchi, lung apices, pleura, pleural cavity, alveolar ducts, alveoli, mainstream bronchi, lobar bronchi, hilum, the lung upper lobe, including the apical segment, posterior segment, and anterior segment, the lung middle lobe, including the medial basal segment and the lateral segment, and the lung lower lobe, including the superior segment, posterior basal segment, and anterior basal segment. Any of the lung lobes or lobe segments may be located on the right or left lung.

Specific formulation to reach a deposit or target tissue may comprise designing the composition to comprise particles having a specific diameter. For instance, formulations comprising particles having a diameter larger than about 5 µm may be designed to reach the bronchial airways. Respiratory mucus secretion can be a barrier to drug delivery. Inhaled particles can trigger mucus secretion in a matter of milliseconds. Mucus secretion is the respiratory system’s first line defense against inhaled particles. In some embodiments, respiratory mucus secretion can be used to enhance drug residence time and absorption through the use of mucoadhesives. For instance, suitable mucoadhesives include carbomer, sodium carboxymethylcellulose, polycarbophil, and sodium alginate. Compositions for delivery to the built environment or environmental surface may be specially formulated to penetrate and reach certain deposit or target tissues.

### Use of Microbiome Compatible Products With Administration of Nonpathogenic Bacteria

Microbiome compatible products may be used in conjunction with the preparations and methods disclosed herein. Various products may be considered to be “biome-friendly” or “biome-compatible.” Examples of biome-friendly products are disclosed in International (PCT) Patent Application Publication No. WO2017/004534 (International (PCT) Patent Application Serial No. PCT/US/2016/040723 as filed on Jul. 1, 2016) which is hereby incorporated herein by reference in its entirety for all purposes. Some biome-friendly products may be cosmetic or therapeutic in nature. In accordance with one or more embodiments, biome-friendly products may be used in combination with microorganisms, e.g., nonpathogenic microorganisms, e.g., beneficial bacteria, which may in turn be used in the form of a preparation or composition to be administered to a subject. Nonpathogenic bacteria disclosed herein may be administered for a cosmetic or therapeutic indication in conjunction with a biome-friendly or biome-compatible product.

In accordance with one or more embodiments, a preparation, composition, formulation or product comprising nonpathogenic bacteria, e.g., for cosmetic or therapeutic use, may itself be considered biome-friendly. In other embodiments, a preparation comprising nonpathogenic bacteria may be used in conjunction with a biome-friendly product. In some embodiments, a preparation comprising nonpathogenic bacteria may be mixed with a biome-friendly product or otherwise administered concurrently. In other embodiments, a preparation comprising nonpathogenic bacteria may be distinct or separate from a biome-friendly product although potentially used in conjunction therewith. In some embodiments, a biome-friendly product is used alone. Nonpathogenic bacterial composition preparations for use in conjunction with a biome-friendly product may be formulated for cosmetic or therapeutic use.

Biome-friendly or biome-compatible products may be used in conjunction with a nonpathogenic bacterial preparation formulated for any mode of delivery, e.g., formulated for targeted delivery to a subject, e.g., to a target tissue, region, system, or organ of a subject. For example, the nonpathogenic bacterial preparation to be used in conjunction with a biome-friendly product may be formulated for delivery to the eye, ear, nose, urogenital system, respiratory system, or gastrointestinal system of the subject. In some embodiments, the nonpathogenic bacterial composition for use with a biome-friendly product may be formulated for targeted delivery based on a condition or disorder of a subject. For instance, the formulation for targeted delivery may be based on a desired local or systemic effect to be achieved, e.g., a local or systemic therapeutic or cosmetic effect.

Biome-friendly cosmetic products that may be used with the present disclosure may be, or include, or be disposed in any one or more of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

Products, e.g., microbiome-compatible cosmetic products, e.g., shampoos, conditioners, and cleansers, as described herein may be used in conjunction with the treatment of a condition, disease, or disorder. These cosmetic products may be used in conjunction with administration of the nonpathogenic bacteria for therapeutic or cosmetic purposes. For example, throughout the treatment period or cosmetic period of time of administering the nonpathogenic bacteria to a subject, the microbiome-compatible cosmetic products may be used. The microbiome-compatible cosmetic products may be used for a period of time prior to commencement of treatment of the therapeutic or cosmetic condition through administration of nonpathogenic bacteria to a subject. The microbiome-compatible cosmetic products may be used for a period of time subsequent to commencement of treatment of the therapeutic or cosmetic condition through administration of nonpathogenic bacteria to a subject. The microbiome-compatible cosmetic products may be used for a period of time subsequent to discontinuation of therapeutic or cosmetic treatment of the condition through administration of nonpathogenic bacteria to a subject.

In some embodiments, the subject may apply one or more cosmetic product, and wait a period of time before administration of the nonpathogenic bacteria. In other embodiments, the subject may administer the nonpathogenic bacteria, and wait a period of time before applying one or more cosmetic products.

The period of time the subject may wait may be about 1 minute, 5 minutes, 10, 15, 20, 25, 30, 45, 60, 90, 120 minutes, or 3 hours, 4, 5, 6, 7, 8, 12, 18, 24 hours after applying one or more cosmetic product and prior to administration of nonpathogenic bacteria.

The period of time the subject may wait may be about 1 minute, 5 minutes, 10, 15, 20, 25, 30, 45, 60, 90, 120 minutes, or 3 hours, 4, 5, 6, 7, 8, 12, 18, 24 hours after administering the nonpathogenic bacteria and prior to applying one or more cosmetic products.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

Certain embodiments are within the scope of the following claims.

1. A method of treating an environmental surface, comprising:
depositing a preparation comprising nonpathogenic bacteria on the environmental surface,
thereby treating the environmental surface.

2. A method of introducing nonpathogenic bacteria into a built environment, comprising:
introducing a preparation comprising the nonpathogenic bacteria to a conduit in fluid communication with the built environment.

3. A method of treating an environmental surface, comprising:
depositing a preparation comprising ammonia oxidizing microorganisms (AOM) on the environmental surface,
thereby treating the environmental surface.

4. A method of modulating a microbiome associated with a built environment, comprising:
conditioning the built environment with a preparation comprising AOM,
thereby modulating the microbiome associated with the built environment.

5. A method of introducing AOM into a built environment, comprising:
introducing a preparation comprising AOM to a conduit in fluid communication with the built environment.

6. A method of maintaining a microbiome associated with a built environment, comprising:
introducing a preparation comprising AOM to a conduit in fluid communication with the built environment.

7. A method of treating a subject, comprising:
introducing an effective amount of a preparation comprising AOM to a built environment of the subject,
thereby treating the subject.

8. The method of any of the preceding claims, wherein the environmental surface is associated with an inanimate object.

9. The method of any of the preceding claims, wherein the environmental surface relates to a surface of a building.

10. The method of any of the preceding claims, wherein the environmental surface relates to a surface of a construction material.

11. The method of any of the preceding claims, wherein the environmental surface relates to an article of furniture.

12. The method of any of the preceding claims, wherein the environmental surface relates to a surface of a fixture.

13. The method of any of the preceding claims, wherein a sufficient amount of the preparation comprising AOM is deposited such that AOM are transferred to a subject, e.g. a subject whose body, e.g., skin, nose, etc., comes into contact with the environmental surface.

14. The method of any of the preceding claims, wherein a target percentage of administered AOM are transferred to the subject.

15. The method of any of the preceding claims, wherein the preparation modulates a microbiome or microbial community associated with or on the environmental surface.

16. The method of any of the preceding claims, further comprising introducing a subject to the built environment.

17. The method of any of the preceding claims, wherein the conduit comprises an auxiliary system, e.g. an HVAC system.

18. The method of any of the preceding claims, wherein the conduit comprises an appliance, e.g. a humidifier or an air conditioner, having a reservoir comprising the preparation of AOM.

19. The method of any of the preceding claims, wherein the auxiliary system or appliance is configured to provide the preparation as a spray, aerosol, or mist characterized by a predetermined particle size.

20. The method of any of the preceding claims, wherein the preparation is introduced as fine particles.

21. The method of any of the preceding claims, wherein the particles have a particle size range (PSR) of between about 0.1 micron and about 500 microns.

22. The method of any of the preceding claims, wherein at least 1, 5, 10, 20, 30, 40, 50, 60, 70, 80, 85, 90, 95, or 99% of the particles have a PSR of between about 0.1 micron and about 10 microns.

23. The method of any of the preceding claims, wherein the preparation is introduced substantially continuously, e.g., substantially continuously during operation of the auxiliary system or appliance.

24. The method of any of the preceding claims, wherein the preparation is introduced periodically.

25. The method of any of the preceding claims, wherein the preparation is introduced to a defined space within the built environment.

26. The method of any of the preceding claims, wherein the built environment is a substantially enclosed space.

27. The method of any of the preceding claims, wherein the built environment is sterilized prior to introduction of the preparation comprising AOM.

28. The method of any of the preceding claims, wherein the environmental surface is sterilized prior to depositing the preparation comprising AOM.

29. The method of any of the preceding claims, wherein the health and/or well being of a subject is promoted.

30. The method of any of the preceding claims, wherein introducing the preparation comprises diffusing the preparation into the built environment.

31. The method of any of the preceding claims, wherein introducing the preparation is associated with a desired local effect, e.g. a desired cosmetic effect or a desired therapeutic effect.

32. The method of any of the preceding claims, wherein introducing the preparation is associated with a desired systemic effect, e.g. a desired cosmetic effect or a desired therapeutic effect.

33. The method of any of the preceding claims, wherein administering the effective amount of the preparation promotes endothelial function.

34. The method of any of the preceding claims, wherein administering the effective amount of the preparation changes or alters a level of nitrite or NO in the subject, e.g. locally or in circulation.

35. The method of any of the preceding claims, wherein administering the effective amount of the preparation modulates a microbiome associated with the subject.

36. The method of any of the preceding claims, wherein the preparation is administered prior to onset of a health condition.

37. The method of any of the preceding claims, wherein the preparation is administered during incidence of a health condition.

38. The method of any of the preceding claims, wherein the preparation is administered subsequent to the subsiding of a health condition.

39. The method of any of the preceding claims, wherein the preparation is administered in response to a health-related symptom, trigger, or warning sign.

40. The method of any of the preceding claims, further comprising determining whether the subject is in need of treatment for a health condition.

41. The method of any of the preceding claims, wherein the health condition is an inflammatory condition.

42. The method of any of the preceding claims, wherein the preparation is administered as a solution, suspension, liquid, gel, or hydrogel.

43. The method of any of the preceding claims, wherein the preparation is formulated as an inhalable solution.

44. The method of any of the preceding claims, wherein the preparation comprises a carrier, e.g., liquid or gas carrier.

45. The method of any of the preceding claims, wherein the preparation is formulated to be substantially bio-compatible.

46. The method of any of the preceding claims, wherein the preparation is formulated to provide a health benefit or otherwise support a healthy lifestyle.

47. The method of any of the preceding claims, wherein the preparation is formulated to provide a health benefit to at least one of the skin, scalp, gastrointestinal system, intranasal cavities, and respiratory system of the subject.

48. The method of any of the preceding claims, wherein the preparation is substantially isotonic.

49. The method of any of the preceding claims, wherein the preparation has a substantially physiological pH level.

50. The method of any of the preceding claims, wherein the preparation is formulated for immediate release or extended release.

51. The method of any of the preceding claims, wherein the preparation is formulated to deliver nitrite or NO to a target tissue or systemically.

52. The method of any of the preceding claims, wherein the preparation is formulated for transmucosal delivery and/or circulation, e.g. locally or systemically.

53. The method of any of the preceding claims, further comprising administering a second amount of the preparation to the subject.

54. The method of any of the preceding claims, wherein the preparation is administered as part of a combination therapy.

55. The method of any of the preceding claims, further comprising administering a second treatment in combination with the preparation.

56. The method of any of the preceding claims, wherein the preparation is administered for a period of time prior to initiating the second treatment.

57. The method of any of the preceding claims, wherein the preparation is administered concurrently with the second treatment.

58. The method of any of the preceding claims, wherein the preparation is administered for a period of time subsequent to ceasing the second treatment.

59. The method of any of the preceding claims, wherein the second treatment is administered via an alternate mode of administration, e.g. orally or intranasally.

60. The method of any of the preceding claims, wherein the subject has a therapeutic level of a second treatment.

61. The method of any of the preceding claims, wherein the preparation is administered in conjunction with a medical approach that treats, e.g., is approved to treat or is commonly used to treat, a relevant disease or disorder, or a symptom of the relevant disease or disorder, e.g., an anti-inflammatory agent or a treatment for infection.

62. The method of any of the preceding claims, wherein the preparation is administered in conjunction with nitrite, nitrate, or NO.

63. The method of any of the preceding claims, wherein the effective amount is a therapeutically effective dose of AOM.

64. The method of any of the preceding claims, wherein the therapeutically effective dose of AOM is about or greater than about 1 × 10³, 10⁴, 10⁵, 10⁶, 10⁷, 10⁸, 10⁹, 10¹⁰, 10¹¹, 10¹², 10¹³, or 10¹⁴ CFU.

65. The method of any of the preceding claims, wherein the preparation is administered as an analgesic.

66. The method of any of the preceding claims, wherein the preparation is administered as a prophylactic.

67. The method of any of the preceding claims, wherein the preparation is self-administered.

68. The method of any of the preceding claims, wherein the preparation is administered about 1, 2, 3, 4, 5, 6, 7, 8, 9, 10, 11, 12, 13, 14, 15, 16, 17, 18, 19, 20, 21, 22, 23, or 24 times per day.

69. The method of any of the preceding claims, wherein the preparation is administered for about 1-3, 3-5, 5-7, 7-9, 5-10, 10-14, 12-18, 12-21, 21-28, 28-35, 35-42, 42-49, 49-56, 46-63, 63-70, 70-77, 77-84, or 84-91 days.

70. The method of any of the preceding claims, wherein the preparation is administered within 30, 60, 90, 120, 150, or 180 minutes of the subject waking from sleep.

71. The method of any of the preceding claims, wherein the preparation is administered within 30, 60, 90, 120, 150, or 180 minutes prior to the subject sleeping.

72. The method of any of the preceding claims, wherein the preparation is administered within 30, 60, 90, 120, 150, or 180 minutes of the subject eating.

73. The method of any of the preceding claims, wherein the preparation is administered 30, 60, 90, 120, 150, or 180 minutes before the subject cleanses or showers.

74. The method of any of the preceding claims, wherein the subject is female.

75. The method of any of the preceding claims, wherein the subject is male.

76. The method of any of the preceding claims, wherein the subject is characterized as one of the following ethnicity/race: Asian, black or African American, Hispanic or Latino, white, or multi-racial.

77. The method of any of the preceding claims, wherein the subject is of an age of less than 1, or between 1-5, 5-10, 10-20, 20-30, 30-40, 40-50, 50-60, or over 60 years.

78. The method of any of the preceding claims, wherein the subject has a disrupted microbiome.

79. The method of any of the preceding claims, wherein the subject is healthy, e.g., physiological, normal, or non-diseased.

80. The method of any of the preceding claims, wherein the subject has a healthy microbiome, e.g., physiological, normal, or non-diseased microbiome.

81. The method of any of the preceding claims, wherein the preparation comprises AOM in a buffer solution, e.g., an aqueous buffer solution.

82. The method of any of the preceding claims, wherein the buffer solution, e.g., aqueous buffer solution, comprises disodium phosphate and magnesium chloride, for example, 50 mM Na₂HPO₄ and 2 mM MgCl₂ in water.

83. The method of any of the preceding claims, wherein the buffer solution e.g., aqueous buffer solution, consisting essentially of disodium phosphate and magnesium chloride, for example, 50 mM Na₂HPO₄ and 2 mM MgCl₂ in water.

84. The method of any of the preceding claims, wherein the buffer solution, e.g., aqueous buffer solution, consists of disodium phosphate and magnesium chloride, for example, 50 mM Na₂HPO₄ and 2 mM MgCl₂ in water.

85. The method of any of the preceding claims, wherein the preparation further comprises or is administered concurrently with a compound that promotes growth or metabolism of the AOM, NO production, and/or urease activity.

86. The method of any of the preceding claims, wherein the preparation comprises at least one of ammonia, ammonium salts, and urea.

87. The method of any of the preceding claims, wherein the preparation comprises a controlled release material, e.g., slow release material.

88. The method of any of the preceding claims, wherein the preparation further comprises an excipient, e.g., a pharmaceutically acceptable excipient.

89. The method of any of the preceding claims, wherein the excipient comprises one or more of: absorption and penetration enhancers, preservatives, antioxidants, buffers, chelating agents, ion exchange agents, solubilizing agents, suspending agents, thickeners, surfactants, wetting agents, tonicity-adjusting agents, enzyme inhibitors, and vehicle for proper drug delivery.

90. The method of any of the preceding claims, wherein the preparation further comprises one or more of antimicrobial agents, inert gases, protectants, vasoconstrictors or vasodilators, vehicle, water, a buffer system, lactated Ringer’s solution, saline, sugar, chelator, and a multi-dose preservative.

91. The method of any of the preceding claims, wherein the preparation is substantially free of other organisms.

92. The method of any of the preceding claims, wherein the preparation comprises between about 1 × 10³ CFU/mL to about 1 × 10¹⁴ CFU/mL AOM.

93. The method of any of the preceding claims, wherein the preparation comprises between about 1 × 10⁹ CFU/mL to about 10 × 10⁹ CFU/mL AOM.

94. The method of any of the preceding claims, wherein the AOM comprise ammonia oxidizing bacteria (AOB).

95. The method of any of the preceding claims, wherein the AOM consist essentially of AOB.

96. The method of any of the preceding claims, wherein the AOM consist of AOB.

97. The method of any of the preceding claims, wherein the AOM comprise Nitrosomonas, Nitrosococcus, Nitrosospira, Nitrosocystis, Nitrosolobus, Nitrosovibrio, and combinations thereof.

98. The method of any of the preceding claims, wherein the AOM is Nitrosomonas eutropha (N. eutropha).

99. The method of any of the preceding claims, wherein the AOM is N. eutropha D23, having ATCC accession number PTA-121157.

100. The method of any of the preceding claims, wherein the AOM comprise ammonia oxidizing archaea (AOA).

101. The method of any of the preceding claims, wherein the AOM are capable of converting ammonia or ammonium to nitrite at a rate of at least about 1 pmol/min/mg protein, e.g., at least about 0.1 nmol/min/mg protein.

102. The method of any of the preceding claims, wherein the nonpathogenic bacteria comprise beneficial bacteria.

103. The method of any of the preceding claims, wherein the nonpathogenic bacteria comprise a selected community of species of bacteria.

104. The method of any of the preceding claims, wherein at least 10, 20, 30, 40, 50, 60, 70, 80, 85, 90, 95, or 99% of the nonpathogenic bacteria in the preparation consist essentially of a selected community of species of bacteria.

105. The method of any of the preceding claims, wherein the nonpathogenic bacteria are selected from a nonpathogenic species in the group consisting of Prevotella, Sphingomonas, Pseudomonas, Acinetobacter, Fusobacterium, Megasphaera, Veillonella, Staphylococcus, Streptococcus, Corynebacterium, Propionibacterium, Rhodococcus, Microbacterium, Bacillus, Lactobacillus, Lactococcus, Streptomyces, Faecalibacterium, Bacteroides, or Bifidobacter, and combinations thereof.

106. The method of any of the preceding claims, wherein at least 1, 5, 10, 20, 30, 40, 50, 60, 70, 80, 85, 90, 95, or 99% of the nonpathogenic bacteria are live.

107. The method of any of the preceding claims, comprising administering AOM and other organisms, e.g., a community of organisms.

108. The method of any of the preceding claims, wherein administering occurs before or after a surgical or diagnostic procedure.

109. The method of any of the preceding claims, wherein a biome-friendly product is used in connection with the administered preparation comprising AOM.

110. A preparation comprising AOM, as recited in any of the preceding claims, for administration to a built environment.

111. A preparation comprising AOM, as recited in any of the preceding claims, for administration to an environmental surface.

112. The preparation of any of the preceding claims, wherein the preparation is packaged for single use.

113. The preparation of any of the preceding claims, wherein the preparation is packaged for multiple use.

114. The preparation of any of the preceding claims, comprising AOM and other organisms, e.g., a community of organisms.

115. A device configured to administer an effective amount of a preparation comprising AOM, as recited in any of the preceding claims, to a built environment.

116. A device configured to administer an effective amount of a preparation comprising AOM, as recited in any of the preceding claims, to an environmental surface.

117. A kit comprising a preparation comprising AOM as recited in any of the preceding claims.

118. An environmental surface, comprising:
a microbial community comprising AOM deposited on the environmental surface according to any of the preceding methods.

119. A built environment, comprising:
a microbial community comprising AOM introduced to the built environment according to any of the preceding methods.
